(19) United States
(12) Patent Application Publication
(10) Pub. No.: US 2026/0090752 A1
(43) Pub. Date: Apr. 2, 2026

(54) CLEANING STATION FOR URINE ANALYSIS DEVICE

(71) Applicant: Withings, ISSY LES MOULINEAUX (FR)

(72) Inventors: Inna NDAW, ISSY LES MOULINEAUX (FR); Matthieu MENANTEAU, ISSY LES MOULINEAUX (FR); Julius DEWAVRIN, ISSY LES MOULINEAUX (FR); Victor GEFFRAYE, ISSY LES MOULINEAUX (FR)

(21) Appl. No.: 19/341,742

(22) Filed: Sep. 26, 2025

(30) Foreign Application Priority Data
Sep. 27, 2024 (FR) 2410351

(51) Int. Cl. A61B 5/20; A61B 5/00

(52) U.S. Cl. CPC A61B 5/207; A61B 5/6891; A61B 2560/0406; A61B 2560/045; A61B 2560/06

(57) ABSTRACT

A cleaning station for a urine analysis device, the cleaning station including a container configured to receive a cleaning fluid, the container being further dimensioned to at least partially receive the urine analysis device, so that the urine analysis device is in contact with the cleaning fluid, and a charger configured to supply power to the urine analysis device when the urine analysis device is at least partially received in the container.

### CROSS-REFERENCE TO RELATED APPLICATIONS

This application claims priority to French Patent Application No. FR2410351, filed September 27, 2024, the entire content of which is incorporated herein by reference in its entirety.

### FIELD

The present invention relates to a cleaning station for a urine analysis device. The invention also relates to an assembly comprising a urine analysis device and such a cleaning station configured to receive the urine analysis device, as well as associated cleaning methods. The urine analysis device comprises a housing configured to be placed entirely within a toilet bowl.

### BACKGROUND

Many biological parameters are reflected in an individual's urine. From a urine sample, for example, it is possible to detect health problems such as urinary tract infection, diabetes or kidney failure. A urine sample can also reflect the quality of a diet, identify a fertile period or pregnancy, and detect the use of drugs or tobacco. In these cases, it makes sense to monitor various biological parameters periodically.

Document WO2021/175909 describes a device that fits onto the wall of a toilet bowl and collects a urine sample prior to optical analysis. The device comprises a measuring station and a cartridge that can be removed and replaced from the measuring station. The cartridge contains urine-reactive reagents such as urine test strips.

Such a urine analysis device placed in a toilet bowl presents a cleaning challenge of the housing following the user's repeated micturition on the housing. In fact, the toilet bowl has a flush that causes water to flow down the length of the bowl. This flow of water tends to pass over and/or behind the housing and therefore does not clean the front face of the housing, which can lead to comfort and hygiene problems (yellowing color of the housing, odor, etc.) for the user.

To this end, document FR2405625 (registration number) proposes an attachment arm comprising a water supply ramp configured to direct a flow of water from a toilet bowl flush towards the front face of the housing in order to clean it after each micturition.

The problem of cleaning also arises for the interior of the measuring station, in which user urine flows repeatedly, leading to potential obstructions due to repeated deposits in the fluidic circuit of the measuring station.

### SUMMARY

The present description proposes to further improve the cleaning of the urine analysis device in order to improve the life of the device and the user experience.

To this end, an aspect of the present description relates to a cleaning station for a urine analysis device comprising a container configured to receive a cleaning fluid, the container being further dimensioned to receive at least in part the urine analysis device, so that the urine analysis device is in contact with the cleaning fluid; a charger configured to supply energy to the urine analysis device when the urine analysis device is received at least in part in the container.

Specifically, the cleaning station allows the urine analysis device to be at least partially received and soaked in the cleaning fluid present in the container, which enables at least part of the device's exterior to be cleaned, particularly around the collection port.

In addition, the charger of the cleaning device supplies energy to the urine analysis device, enabling, for example, the recharging of a rechargeable battery of the device before a new cycle of use in the user's toilet and thus recharging the battery during a cleaning phase. The recharging and cleaning tasks are carried out at the same time, which reduces the downtime of the urine analysis device.

This power supply via the charger can also be used to activate a pump arranged in the device to draw in cleaning fluid and thus clean at least part of the device's fluidic circuit. The cleaning station thus uses the pump of the urine analysis device to clean the fluidic circuit.

The cleaning station according to the present description thus enables easy and effective cleaning for the user, as well as a power supply in parallel with the cleaning. In particular, the charger can be a wireless charger (typically by induction), which simplifies the placement of the urine analysis device in the cleaning station (fewer user actions, no risk of water infiltration or short-circuiting, etc.).

In an embodiment, the container removably holds the urine analysis device.

In an embodiment, the cleaning fluid is a detergent.

In an embodiment, the container comprises a gauge indicating the recommended volume of cleaning fluid to be poured into the container.

In an embodiment, the station is devoid of means for circulating the cleaning fluid in the container.

In an embodiment, the charger is positioned, along the depth of the container, at least partly between the bottom of the container and the gauge.

In an embodiment, the urine analysis device comprises a rechargeable battery and the charger is configured to electrically charge the battery.

In an embodiment, the charger is a wireless charger, so that charging between the charger and the urine analysis device is wireless.

In an embodiment, the charger is electromagnetically inductive.

In an embodiment, the station comprises a coil placed opposite a container wall.

In an embodiment, the coil is placed in a protrusion formed in the wall.

In an embodiment, the station comprises a charging port connectable to the urine analysis device.

In an embodiment, the station comprises an electrical connection cable for connecting the charging system to an electrical network.

In an embodiment, the station comprises a battery.

In an embodiment, the container has a straight cylindrical shape.

In an embodiment, the container has a truncated cone shape.

In an embodiment, the container has an oval, round or square base.

In an embodiment, the urine analysis device has a dimension of lesser length along a direction X, the container being configured to receive the urine analysis device such that the direction X extends horizontally.

In an embodiment, the width of the container along direction X is substantially equal to the dimension of least length.

In an embodiment, the container is configured to receive the device so that the X axis extends along a direction forming an angle of between 20° and 70°, for example approximately 45°. The term “about” or “approximately,” as used herein, is intended to encompass variations of ±10% of the stated value unless otherwise specified. Thus, “approximately 45°” encompasses angles within the range of 40.5° to 49.5°.

In an embodiment, the container comprises at least one protrusion configured to support the urine analysis device.

In an embodiment, the device is solely supported by the protrusion(s) in the container.

In an embodiment, the at least one protrusion defines a clearance volume intended to receive the collection port of the analysis device.

In an embodiment, at least one protrusion is arranged at the bottom of the container.

In an embodiment, the container comprises at least one lateral protrusion projecting substantially horizontally from the wall.

In an embodiment, at least one protrusion is in the form of a promontory configured to support the device.

In an embodiment, the cleaning station comprises a cover configured to at least partially close the container.

In an embodiment, the cover comprises at least one wedge projecting from the cover towards the container, each wedge being configured to hold the urine analysis device in position in the container.

In an embodiment, the cover comprises a hook configured to cooperate with a means of securing the urine analysis device.

In an embodiment, the cover comprises at least one aperture suitable for being passed through by an attachment arm of the urine analysis device.

In an embodiment, the cover is made of an at least partially transparent or translucent material.

In an embodiment, the cover comprises a zone of reduced thickness to make visible a luminous indicator of the device, in particular a luminous charge indicator.

The present description also relates to an assembly comprising :

a urine analysis device, the urine analysis device comprising a housing and a fluidic circuit housed within the housing and configured to circulate urine through the urine analysis device, the fluidic circuit comprising a collection port on the housing,

a station as defined above, the assembly being configured to switch from an operational configuration in which the urine analysis device is received in the container to a separate configuration in which the urine analysis device is away from the container.

In an embodiment, the device is configured to be placed entirely within a toilet bowl in the separate configuration.

In an embodiment, the station and device are configured to be placed entirely within the toilet bowl in the operational configuration.

In an embodiment, the station comprises means for securing the container to the toilet bowl.

In an embodiment, the container is sized so that the collection port is immersed in the cleaning fluid in the operational configuration.

In an embodiment, the container is formed by a wall and a bottom, the container being dimensioned so that the collection port is spaced from at least one of the wall and the bottom.

In an embodiment, the urine analysis device further comprises an arm for attachment to a toilet bowl, the attachment arm projecting at least in part from the container in the operational configuration.

In an embodiment, the fluidic circuit comprises a pump configured to draw liquid into the fluidic circuit via the collection port.

In an embodiment, the charger is configured to, in the operational configuration, power the pump of the fluidic circuit, so that cleaning fluid is drawn into the fluidic circuit.

In an embodiment, the charger is further configured to recharge a battery of the urine analysis device.

In an embodiment, the urine analysis device is configured to switch from a urine analysis configuration to a cleaning configuration, the device comprising at least one reagent suitable for reacting with urine, the fluidic circuit comprising a drainage port on the housing, the fluidic circuit being configured so as, in the urine analysis configuration, to inject the fluid collected by the collection port onto the reagent, the fluidic circuit being configured so as, in the cleaning configuration, to circulate the fluid collected by the collection port directly towards the drainage port.

In an embodiment, the urine analysis device is in the draining position during the operational configuration.

In an embodiment, the fluidic circuit comprises an injector configured to inject urine onto reagent, wherein in the cleaning configuration, the fluidic circuit comprises in series the collection port, the injector and the drain port.

In an embodiment, the device is configured to activate the pump when predetermined cleaning conditions are met.

In an embodiment, the cleaning conditions comprise: receipt of a signal sent by a power supply system of the device indicating that it is receiving power from the charger, and/or receipt of a signal sent by a radar sensor indicating that the container wall is detected, and/or receipt of a signal sent by a temperature sensor indicating that a temperature change on the housing has been detected.

In an embodiment, the urine analysis device is configured to aspirate and purge cleaning fluid present in the container in the operational configuration.

In an embodiment, the device comprises a radar sensor configured to detect the presence of at least one wall in the vicinity of the device in the operational configuration.

In an embodiment, the device comprises a temperature sensor configured to detect a change in temperature at the container in the operational configuration.

In an embodiment, the fluidic circuit comprises a liquid sensor configured to detect a presence of liquid in the fluidic circuit in the operational configuration, in response to pump activation.

The present description also relates to a cleaning and charging method implemented by an assembly as defined above, the method comprising the following successive steps, after setting the assembly from the separated configuration to the operational configuration:

powering the device by the charger,

cleaning the device with the cleaning fluid in the container.

In an embodiment, the cleaning is the cleaning of at least part of the outer wall of the case.

In an embodiment, the cleaning is the cleaning of the fluidic circuit.

In an embodiment, cleaning comprises: activating the pump to circulate cleaning fluid in the fluidic circuit.

In an embodiment, the pump is powered by energy received from the wireless charger.

In an embodiment, the cleaning method comprises a cleaning phase during which:

the device receives energy from the charger to power the pump in particular,

the pump is activated, so that liquid is drawn through the collection port.

The present description also relates to a urine analysis device comprising: - a housing,

a fluidic circuit housed inside the housing, comprising a collection port, a drainage port and a pump configured to circulate urine in the urine analysis device,

a power supply system configured to supply power to the pump, the power supply system being rechargeable by an induction charger, wherein the urine analysis device is configured to selectively be : in a urine analysis configuration during which the fluidic circuit is configured to inject urine onto a urine reagent, or in a cleaning configuration during which the collection port is connected to the drainage port, wherein the fluidic circuit is configured to, in the cleaning configuration, circulate the fluid collected by the collection port directly to the drainage port, wherein the device is configured to :

detect that the power supply system is in contact with a wireless charger and,

in response to detection, switch the device to a cleaning configuration.

In an embodiment, there is provided a cleaning station for a urine analysis device having a housing, a collection port, and a pump, the cleaning station comprising: a container having a wall and a bottom that define an internal volume accessible through an opening and configured to receive a cleaning fluid; at least one support feature arranged within the internal volume to position a received urine analysis device such that the collection port is immersed in the cleaning fluid and lies within a clearance pocket spaced from the wall and/or the bottom by at least 3 mm to permit unimpeded intake of the cleaning fluid through the collection port; and a wireless charger comprising an inductive coil disposed in or adjacent the wall, the charger being positioned along a depth of the container at least partly between the bottom and a fill gauge and being configured to transfer power to the received urine analysis device while the collection port is immersed; wherein the cleaning station lacks any pump configured to circulate the cleaning fluid within the container, so that circulation through the device occurs by operation of the pump of the received urine analysis device when powered by the charger.

In one or more embodiments, a “support feature” refers to any protrusion or structural element arranged within the container and configured to hold, stabilize, and orient the urine analysis device in the operational configuration. The support feature may be formed, for example, as one or more bottom-mounted protrusions acting as a cradle, as one or more lateral protrusions projecting from the container wall to wedge the device laterally, or as a promontory having a receiving surface shaped to complement a face of the housing. In certain embodiments, the support feature further includes a retaining element, such as a notch, that cooperates with a portion of the housing or its attachment element to prevent movement of the device. Beneficially, the support feature is configured so that the urine analysis device is supported solely by the feature and not in direct contact with the remaining container wall, while positioning the collection port within a clearance volume spaced from the container wall and/or bottom to ensure immersion in the cleaning fluid and unimpeded fluid intake.

### BRIEF DESCRIPTION OF THE DRAWINGS

Further features, details and benefits will become apparent from the detailed description below, and from an analysis of the appended drawings, in which:

FIG. 1 shows a simplified schematic representation of a urine analysis device installed in a toilet bowl,

FIG. 2 shows an exploded view of the urine analysis device, in which the measuring station and cartridge are visible,

FIG. 3 shows a detailed view of a cartridge according to one embodiment in cooperation with an actuator of the measuring station,

FIG. 4 shows a sectional view of a cartridge and a measuring station according to one embodiment, at the location of an optical analyzer of the measuring station,

FIG. 5 shows a side view of the urine analysis device with an attachment arm,

FIG. 6 shows a perspective view of a cleaning station according to the present description,

FIG. 7 shows a vertical cross-sectional view of the cleaning station, in separate configuration (a) without an inserted urine analysis device and in operational configuration (b) with an inserted urine analysis device,

FIG. 8 shows a horizontal cross-sectional view of the cleaning station, in separate configuration (a) without an inserted urine analysis device and in operational configuration (b) with an inserted urine analysis device,

FIG. 9 shows a perspective view of a cleaning station cover,

FIG. 10 shows a perspective view of an assembly in operational position, with the device with the attachment arm inserted in the cleaning station,

FIG. 11 shows a perspective view of an alternative version of the cleaning station cover,

FIG. 12 shows a front view of an assembly in a separate configuration, with the device attached to the cover away from the cleaning station,

FIG. 13 shows a perspective view of an alternative cleaning station (a) and a vertical sectional view of this cleaning station (b),

FIG. 14 shows a perspective view of the assembly in operational configuration, with the cleaning station of FIG. 13 and the urine analysis device inserted in the cleaning station,

FIG. 15 shows a side view of another embodiment of an assembly in operational configuration, in the toilet bowl,

FIG. 16 shows schematically a view of the components of the device according to one embodiment of the description, as well as its ecosystem.

### DETAILED DESCRIPTION

The present description presents various examples of a urine analysis device comprising a station and a cartridge as disclosed in documents WO2021175909, WO2021175944, WO2023036805, WO2023036806, WO2023036808, WO2023036809.

The following paragraphs explain the general principle of a urine analysis device, but all the details of the WO documents in the previous paragraph are applicable.

### General shape of the housing

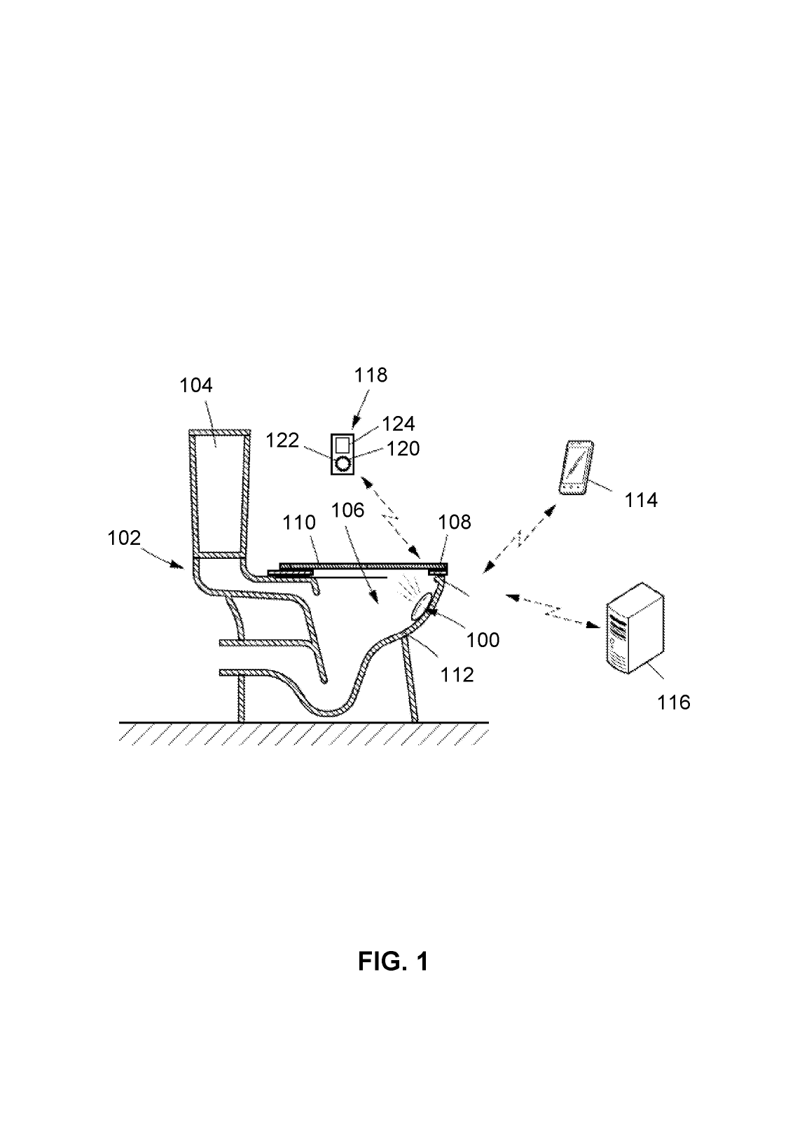

FIG. 1 schematically illustrates an analysis device 100 (hereinafter also referred to as "device 100") for urine analysis installed in a toilet 102. Toilet 102 typically comprise a water tank 104, a bowl 106, a seat 108 and a seat cover 110. The analysis device 100 is configured to be placed entirely within the toilet bowl. By "in the bowl" is meant "placed in the interior volume defined by the bowl". The analysis device 100 is removably arranged in the toilet 102. For example, the analysis device 100 can be easily removed from the toilet to replace a cartridge, then reinstalled in the toilet 102. The analysis device 100 is placed on an inner wall 112 of the toilet bowl 106. The analysis device 100 is positioned so that it is generally under a user's urine stream, so that when a user urinates (generally in a seated position), the urine comes into contact with the analysis device 100. The analysis device 100 can communicate remotely with a remote entity, such as the smartphone 114 or a server 116.

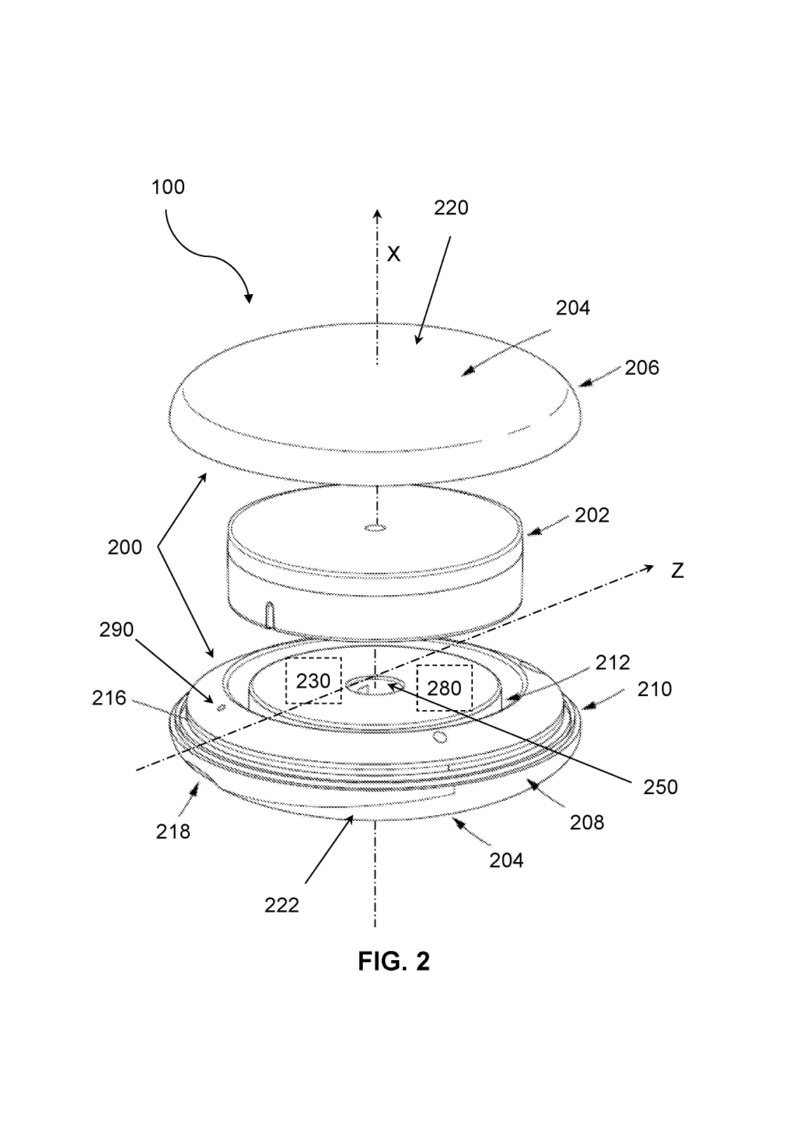

As illustrated in greater detail in FIG. 2, the analysis device 100 may comprise a measurement station 200 and a cartridge 202, removably mounted on the measurement station 200. Cartridge 202 contains reagent which can react with urine (also known as "urine reagent"). Alternatively, the measuring station 200 can be fitted directly with urine reagent, without any removable parts. Alternatively, the measuring station 200 can be recharged by pouring in reagent.

Measuring station 200 may comprise a housing 204 which may comprise two shells, in particular a front shell 206 and a rear shell 208. The front shell 206 and the rear shell 208 may cooperate with each other via a fastening mechanism 216, in a plane normal to the X axis. The front shell 206 and the rear shell 208 can be reversibly assembled, for example by screwing or clipping. Alternatively, the front shell 206 and rear shell 208 can be permanently joined, for example by gluing, clipping, magnetizing or ultrasonic welding. Of course, other fastening means can be used to join the two shells.

In particular, as shown in FIG. 2, the front shell 206 and the rear shell 208 are screwed together. Then, an internal part of the front shell 206 comprises a thread. The thread on the front shell 206 is designed to cooperate with a complementary thread on the rear shell 208. This allows the housing 204 to be easily disassembled to gain access to the test assembly inside the housing.

A seal may be present between the front shell 206 and the rear shell 208. In this way, housing 204 is watertight. Only collection and drainage ports connect the outside and inside of housing 204, as described in more detail below.

As can be seen from the figures, housing 204 can have the overall external shape of a circular roller. In other words, the housing 204 has a spheroidal shape. The X axis is the center line of the housing. Beneficially, the front shell 206 can be substantially rotationally symmetrical, giving the device an aerodynamic appearance once installed. Housing 204 serves as a urine collector.

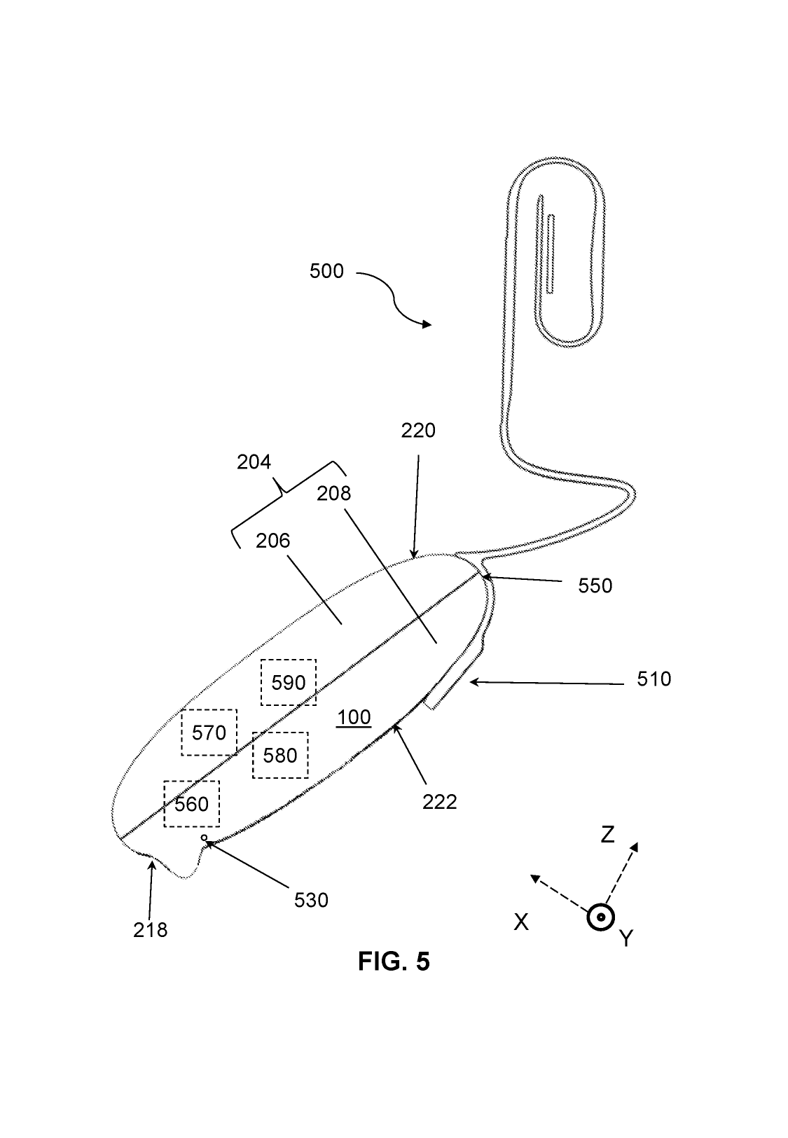

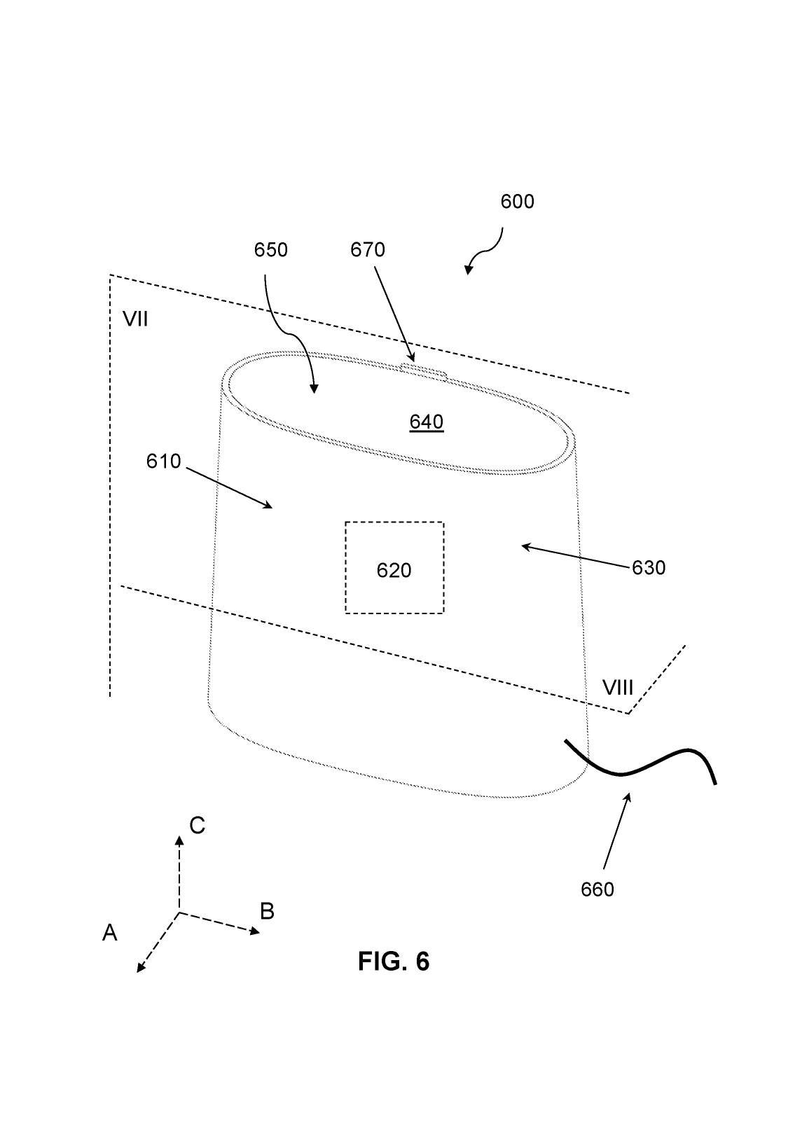

The housing 204 comprises a front face 220 for receiving a stream of urine directly from a user urinating on the toilet, and a rear face 222 opposite the front face 220. As illustrated in FIG. 2, the front face 220 can be arranged on the front shell 206 and the rear face 222 can be arranged on the rear shell 208. The front face 220 faces the inside of the bowl 106. The front face 220 is therefore intended to receive urine when the user urinates while sitting on the toilet 102. As shown in FIGS. 5 and 6, the rear face 222 faces the inner wall 112 of the bowl 106. For the rest of the description, an object facing the bowl wall is taken to mean an object facing the bowl wall closest to the object in question, and not the facing bowl wall on the other side of the inner bowl volume.

The front face 220 and rear face 222 each have a curved edge 210. The respective curved edges 210 of the front and rear faces meet at an equatorial junction zone. Thus, the outer surface of housing 204, consisting of front face 220 and rear face 222, is defined by curved lines and forms a generally convex object.

The outer surface of the front face 220 may be smooth. In other words, the front face 220 is free of ridges or grooves. In this way, the flow of urine coming into contact with the front face 220 catches and spreads over the front face 220. The front face 220 may be substantially rotationally symmetrical about the X axis.

The outer surface of housing 204 may also be white or light-colored. The color of the outer surface can be similar to that of the toilet, which enhances the discreetness of the device.

In an embodiment, housing 204 may have a diameter, measured in the direction orthogonal to the X axis, of between 50 mm and 150 mm. In an embodiment, the housing 204 can have a thickness, measured in the direction of the X axis, of between 15 mm and 50 mm. In this way, housing 204 is sufficiently compact to be completely housed in the toilet bowl. The urine analysis device 100 is unobtrusive. In addition, housing 204 is large enough to systematically come into contact with the urine received in the toilet bowl. The user can then urinate in the toilet without worrying about the urine analysis device, or alternatively aim generally in its direction.

According to another aspect, In an embodiment, the housing 204 has a general form factor such that the ratio between thickness and diameter is between 0.2 and 0.5, and for example between 0.3 and 0.4. Such proportions are reminiscent of a natural pebble and give the device a soothing appearance. The spheroidal ‘pebble’ shape minimizes splash-back and offers low resistance to water flow, encouraging complete and uniform flushing.

Housing 204 may be made of a hydrophilic material. For example, the material of housing 204 may be a ceramic, a polyamide (PA), a silicone or a hydrophilic polymer. The outer surface of housing 204 can also be treated with a hydrophilic surface treatment, e.g. Aculon's acuWet®, a hydrophilic polymer, or Arkema's Pebax®.

### Fluidic circuit

A fluidic circuit 230 is present inside housing 204 and configured to perform an analysis of the collected urine. Measuring station 200 comprises an annular compartment 212, located inside housing 204, arranged around an axis of rotation X. The annular compartment 212 is configured to at least partially receive the cartridge 202 mounted rotatably around the axis of rotation X (once in position in the annular compartment 212). The cartridge 202 comprises a plurality of test holders each comprising at least one urine reagent, for example a dry reagent, the plurality of test holders being arranged along a circle or arc of a circle around the axis of rotation X. In an embodiment, the test carriers are test strips. The test carriers can be enclosed, for example individually, in a chamber.

The annular compartment 212 typically extends over 360° and forms a groove configured to at least partially receive the cartridge 202.

The measuring station 200 includes a collection port 218, located for example on the rear shell 208. Collection port 218 is configured to collect urine flowing onto the surface of housing 204. The measuring station 200 also includes a drain port 530, visible in FIG. 5, configured to drain the collected liquid out of the device 100. The rear-facing collection port and spacer arrangement prevents direct exposure to user urine streams and flush surges, reducing fouling risk and ensuring sensor longevity. This also avoids turbulent flow disruption during sample intake, improving test accuracy.

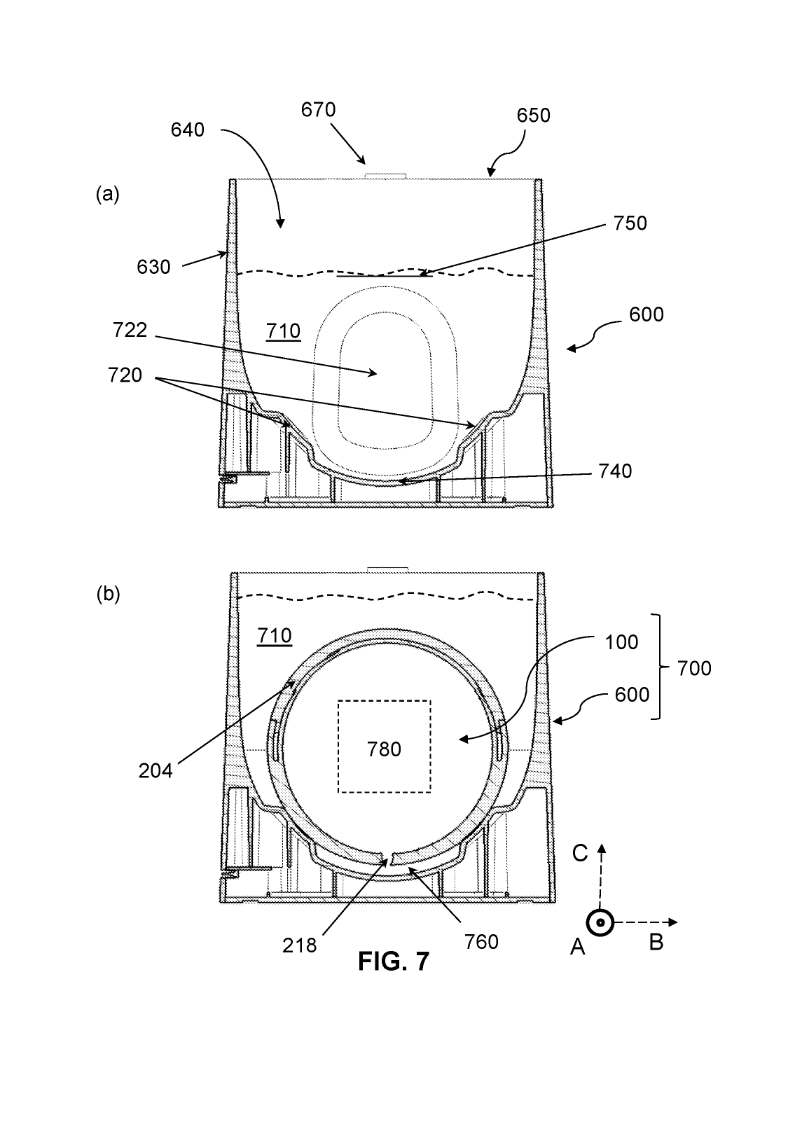

Fluidic circuit 230 may comprise a pump 780 (shown schematically in FIG. 7), an injector and an analyzer 400. In a collection configuration, pump 780 draws liquid, notably urine, from collection port 218. Then, in an injection configuration, the injector injects the urine onto one or more test carriers of the cartridge, and the analyzer obtains certain property values (for example, physical/chemical properties, such as color) from the test carriers after they have come into contact with the urine. In an embodiment, the analyzer is an optical analyzer configured to analyze the optical properties of the test media. The injector and cartridge can move relative to each other so that the injector can open (e.g. pierce) the chamber, for example using a needle or needle-like device.

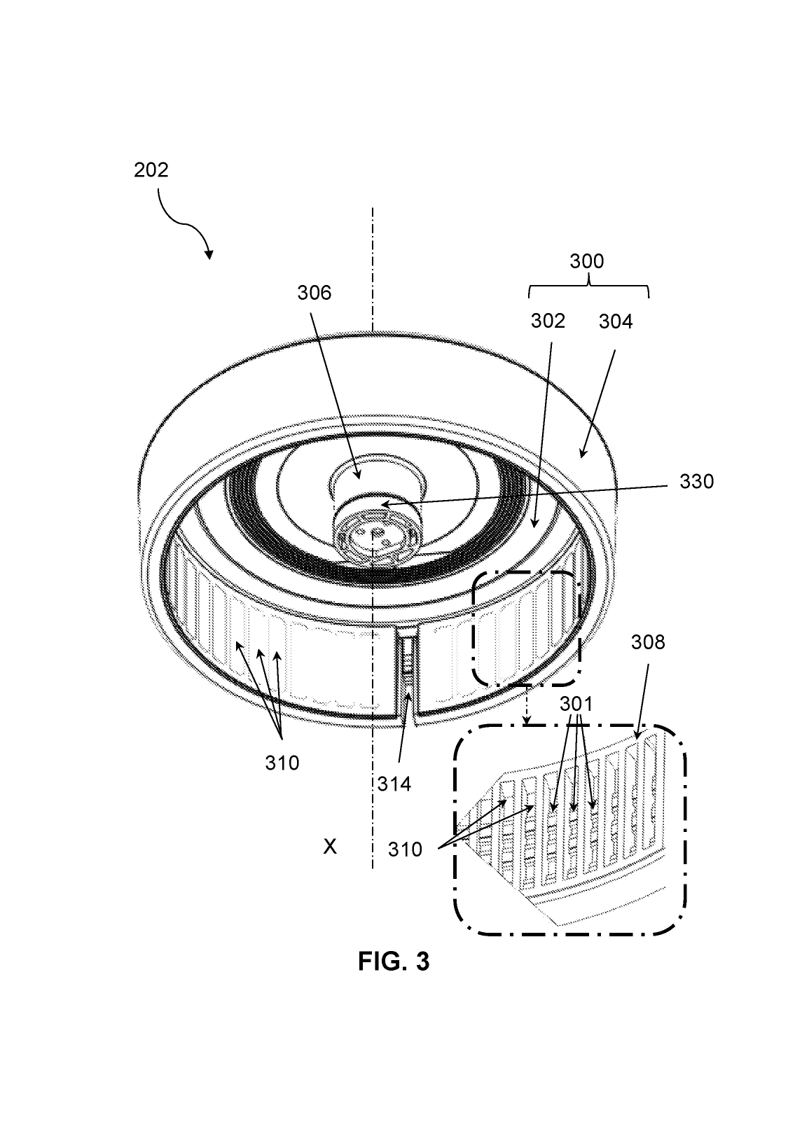

FIG. 3 shows an exploded view of cartridge 202. Cartridge 202 comprises at least one test support 301, in particular a plurality of test supports 301 configured to receive urine from the injector. Each test support 301 contains a urinary reagent which reacts in a specific way on contact with urine. The cartridge 202 comprises a rotating support 300, configured to be rotated by the measuring station 200. During normal use of the cartridge 202 and the device 100, the test supports 301 remain attached to the rotating support and do not move relative to it.

In an embodiment, the rotating support 300 has a straight circular cylinder shape of at least 80% of a hollow cylinder shape extending annularly around an axis which is, when the cartridge 202 is mounted in the measuring station 200, the axis of rotation X. Each test support 301 may be a test strip. The rotating support 300 may comprise an annular portion 302 and a cylindrical portion 304, which extends from a radially outer end of the annular portion 302. The cylindrical portion 304, when in use, is housed inside the annular compartment 212. The test support 301 are positioned along the cylindrical portion 304, so as to be able to move selectively and/or successively past the injector and analyzer. For example, the test support 301 are part of a holder 308, which comprises several chambers 310, separated from each other along a perimeter around the X axis. At least one test strip is received in a chamber 310.

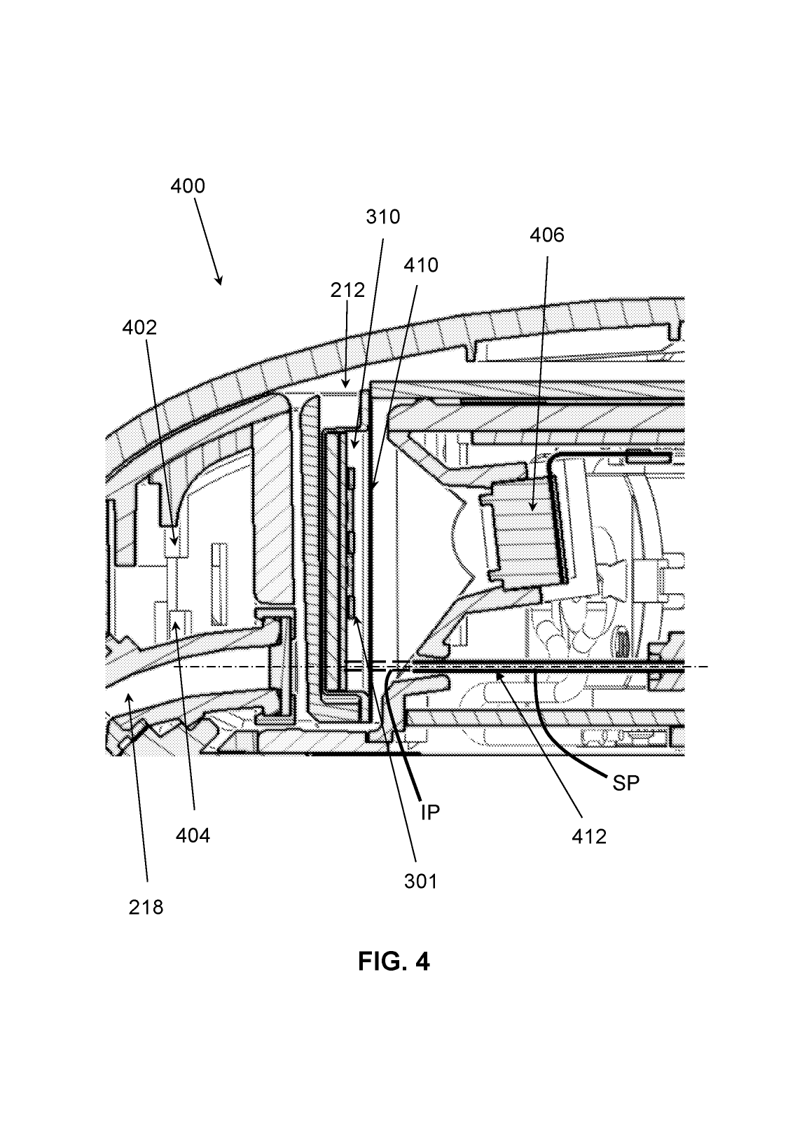

The chambers 310 are arranged side by side in the shape of a right circular cylinder of at least 80% of the circle. To allow light to pass through, the holder 308 comprises at least one opening 312 per chamber 310 (shown in the upper left zoom where the rotating holder is represented as transparent). The chambers 310 are all equidistant from the axis of rotation X, so that the injector can selectively inject urine once the desired chamber is positioned at the desired location facing the injector. The injector can move towards chamber 310 and pierce a lid 410 closing chamber 310 (visible in FIG. 4). In an embodiment, the lid 410 is transparent or translucent to allow the passage of light inside the chamber and/or to perform optical analysis of the reagent, and thus enable analysis of the result of the reagent's reaction with urine. A drain hole 314 is provided in the rotating support 300 to allow urine to be evacuated from the injector to the outside of the device 100, via the drain port 530 located on the housing 204.

The annular portion 302 of the rotating support 300 remains outside the annular compartment 212 to reinforce the cylindrical part and/or rotate the cartridge 202. To this end, the annular portion 302 may comprise a mechanical coupling 306, which cooperates with an actuator 330 of the measuring station 200.

Dimensions relating to cartridge 202 are disclosed in documents WO'909, WO'933 and WO'80X. The maximum dimension of the device 100 transverse to the axis of rotation X is less than 15 cm, or even less than 10 cm. The maximum dimension of the device along the axis of rotation X is less than 5 cm.

FIG. 4 shows in more detail the interaction between cartridge 202 and measuring station 200 when or after injector activation. The analyzer 400 comprises at least one light source 402, 404 (for example, two light sources; in particular, four light sources) and at least one optical sensor 406. Light travels from the light source 602, 604 to the optical sensor 606, passing through the cartridge 202 and in particular the cylindrical part 304 and the test support 301.

In an embodiment, the analyzer 400 is configured to measure the absorbance of a portion of the test supports 301 (in particular the test line and/or the control line of a strip as will be explained later). Absorbance is detected by the light source (e.g. an LED), which can pass light through the strip, and the optical sensor, which receives the spectrum with around ten wavelengths.

In an embodiment, the light sensor is a camera capable of detecting a change in color, in particular a change in color intensity, of a portion of the test support 301 (in particular the test line and/or the control line of a strip as will be explained below). The camera can detect color in RGB values, for example.

The injector comprises an injection end 412 (e.g. a needle), which can be moved between a waiting position SP and an injection position IP. In the standby position SP, the injection end 412 is outside the cartridge 202 (in a radially innermost position), so that the cartridge 202 can rotate freely in the annular compartment 212. In an injection position IP, injection tip 412 has pierced lid 410 to access the interior of chamber 310 and can inject a small amount of urine onto the reagent in test support 301.

In the SP position, the injector is located radially inside the annular chamber. This maximizes the radius of the annular chamber while minimizing the size of the measuring station 200.

In an embodiment, the injector can be moved into a collection configuration in which the injection end 412 is positioned at the collection port 218 to enable collection of a liquid.

In an embodiment, the injector can be moved into a purge configuration in which the injection end 412 is positioned at the drain port 530 to allow liquid to be purged.

Reference is made to documents FR2410306, FR2410307 and FR2410308 (filing numbers) for further details on the operation of the injector.

### Collection port

The collection port 218 is configured to receive a liquid present opposite the port. In particular, collection port 218 is configured to receive urine that flows by gravity onto the outer surface of housing 204 when the device is in position in a toilet. Urine is collected directly on the front face 220 and rear face 222 of housing 204. As will be explained in more detail later, the collection port 218 also collects a cleaning fluid.

The collection port 218 is an opening configured to collect the liquid, so that the liquid can enter the urine analysis device. The collection port 218 is generally circular, with a diameter for example between 0.3 mm and 2 mm. The diameter of the collection port can be chosen to maximize the volume of urine collected on the outer surface of housing 204.

As can be seen from the figures, the collection port 218 is located on the rear face 222. In this way, the collection port 218 faces the inner wall 112 of the toilet when the urine analysis device 100 is positioned in the toilet. This position allows the collection port 218 to be hidden from the user's view by the front face 220 of the housing. The front face 220 visible to the user resembles a simple, uniform pebble, as already mentioned, with no singular points or holes. It should also be noted that this position prevents the introduction of contaminants or elements that could obstruct the fluidic circuit 230.

The collection port 218 is located on a lower part of the rear face 222. By "on a lower part of the rear face", it is meant "on the last quarter of the face along the Z direction from the lower end of the housing 204". The “Z direction” refers to the vertical axis when the device is positioned in its intended use configuration within toilet bowl 106, with the “bottom” defined as the portion of the case oriented toward the base of the bowl and the “top” defined as the portion oriented toward the toilet seat 108. The “lower end” of the case therefore faces the bowl bottom during use and is opposite the uppermost edge of the case. The lower end faces the bottom of bowl 106 when housing 204 is positioned in the bowl. The lower end is opposite the top 550. This position corresponds to normal use. This position enables urine to be collected by gravity over most of the outer surface of housing 204. Locating collection port 218 in this lower region enables urine flowing by gravity across the external surface of housing or case 204 to converge and be captured efficiently. This geometry ensures that the port is positioned at a natural collection point for liquid accumulation during urination. In some embodiments, the port may be located within 20 mm of the bottom edge of the rear face 222, thereby maximizing gravitational collection. In alternative embodiments, the collection port may be placed exactly at the bottom edge itself to ensure immediate capture of downward-flowing liquid. This placement also reduces the risk of miscollection from incidental splashes at higher elevations and ensures that substantially all urine that has contacted the outer surface is directed toward the port. By locating the port on the rear face (facing the bowl wall 112), the opening is concealed from a user’s line of sight, preserving discretion, while still positioned for optimal fluid capture by gravity. For purposes of clarity, “collection port” as used here refers to any defined opening, orifice, or aperture on housing or case 204 through which urine enters the internal reservoir or fluidic circuit, optionally covered by a filter or septum. The term excludes decorative recesses or non-functional features of the case.

In particular, the distance separating the collection port 218 from a lower edge of the housing 204 is less than 40 mm, for example less than 20 mm. As illustrated, according to a particular embodiment, the collection port 218 is arranged a few millimeters above the lower edge of the housing 204. Alternatively, the collection port 218 may be located on the lower edge, the lower edge being defined when the device 100 is placed for use in the toilet.

Collection port 218 may be covered by a mesh filter. The mesh filter is, for example, oblong in shape and covers the collection port 218. The average mesh size of the filter is, for example, 20 microns. The mesh filter prevents the introduction of contaminants or elements likely to obstruct the fluidic circuit 230 and filters the urine received in the collection port 218. The filter mesh can be made of metal.

### Power supply system

The urine analysis device 100 comprises a power supply system 280 configured to supply power to the device 100, and in particular to the electronic components of the device 100. The power supply system 280 can be further configured to store energy.

The power supply system 280 may comprise a rechargeable battery. The rechargeable battery is configured to store chemical energy and to convert this chemical energy into electrical energy. The battery is configured to be recharged several times, i.e. by reversing the electrochemical reaction to recharge the chemical energy stored in the battery. To this end, device 100 is configured to cooperate with a station charger as will be explained below. The battery may be, for example, a Lithium Ion battery or a Lithium Polymer battery.

As will be explained in more detail later, the power supply system 280 can be configured to supply power to the electronic components of the device 100, in particular the pump 780, in parallel with the electrical recharging of the rechargeable battery by a charger.

The urine analysis device 100 may comprise a luminous indicator 290, in particular a luminous charge indicator. The luminous indicator 290 is configured to provide visual information relating to the state of charge of the battery. The indicator light 290 is, for example, an LED configured to light up green when the battery is at least 25% of its charge, orange when the battery is at least 5% of its charge and red when the battery is below 5%.

### Temperature sensor

Device 100 may include a temperature sensor 560 configured to detect a change in temperature at housing 204. The temperature sensor 560 is mounted in the housing 204, for example at the collection port 218.

For example, when urine at a temperature above 35°C drips onto the housing, the temperature sensor 560 is configured to detect a sudden rise in temperature. The temperature sensor 560 is then configured to associate this sudden rise in temperature with the presence of a stream of urine on the housing 204, and for example to trigger the pump 780.

In another example explained in more detail later, when the device 100 is placed in a cleaning fluid present in a container, the temperature sensor 560 is configured to detect a change in temperature on the housing, in particular a drop in temperature, and send a signal to control circuitry of the device 100.

### Radar sensor

Device 100 can include a radar sensor 570 configured to detect the presence of bodies in the vicinity of device 100. To this end, the radar sensor 570 is configured to send a radar signal and to receive at least one reflected radar signal, the reflections being caused by the nearby body or bodies. As used herein, a “radar sensor” refers to an electronic sensing component that operates by emitting electromagnetic waves, typically in the radio frequency (RF) or millimeter-wave (mmWave) spectrum, and by receiving one or more reflected signals that return after contacting an object. By analyzing the time delay, frequency shift, amplitude, or phase of the reflected radar signal, the radar sensor 570 can determine the presence, position, distance, motion, or other properties of the detected object. In this context, the radar sensor 570 provides a non-contact, robust sensing mechanism that can function reliably even in the humid or wet conditions of a toilet bowl environment. By processing the signals received, it is possible to determine the presence and characterize these bodies. For example, radar sensor 570 is configured to detect a stream of urine and determine at least one property relating to this stream of urine. Alternatively or additionally, the radar sensor 570 is configured to detect the genitals of a toilet user, in particular for identification purposes.

Alternatively or additionally, the radar sensor 570 is configured to detect at least one wall in the vicinity of the device 100, in particular opposite the front face 220 of the housing 204. Beneficially, the radar sensor 570 is configured to detect that the device 100 is surrounded by a wall in its vicinity. As will be explained later, the radar sensor 570 can therefore detect that the device 100 has been introduced into a container. This detection may be based, for example, on the radar sensor recognizing characteristic reflection patterns from the container wall at known distances. Accordingly, the radar sensor 570 can be used as a trigger condition for automatically switching the device 100 into a cleaning configuration, as will be explained later.

Reference is made to documents FR3140448 (publication number), FR2403439 and FR2405542 (filing numbers) which describe the implementation of a radar sensor in the urine analysis device.

### Liquid sensor

Device 100 may comprise a liquid sensor 580 configured to detect a presence of liquid in fluidic circuit 230, particularly in response to activation of pump 780. The liquid sensor 580 is formed, for example, by at least two probes at two separate positions in the fluidic circuit. In particular, each probe is configured to identify the state (liquid, gaseous or mixed liquid/gas) of the fluid flowing past the probe. In certain embodiments, the liquid sensor 580 operates by measuring changes in electrical conductivity, capacitance, or impedance between the probes, such changes being indicative of whether the medium present is a liquid or a gas. Alternatively, the sensor may employ optical detection (e.g., light transmission or reflection) or ultrasonic detection to identify the presence of liquid within the circuit. The liquid sensor 580 can also be configured to determine the flow rate of the fluid pumped through the collection port 218. For example, by measuring the transit time of a liquid front between two probes or by monitoring frequency variations in the received signal, the liquid sensor 580 may provide quantitative flow data. Beneficially, this information can be used not only to confirm the presence of cleaning fluid during a cleaning phase, but also to monitor urine sample collection during an analysis phase, thereby improving reliability and reducing the risk of incomplete or false measurements.

Reference is made to document WO2022184984, which describes the operation of such a liquid sensor 580 in greater detail.

### Mounting

Housing 204 is designed to be placed on the inner wall 112 of bowl 106. The housing 204 is fixed by a fastening arm 500. The attachment arm 500 is configured to secure the urine analysis device 100 in the toilet bowl 106. Fastening arm 500 is typically molded from a single material, such as plastic.

The analysis device 100 comprises an attachment element 510 arranged on the rear face 222 of the housing. The attachment element 510 is configured to cooperate with an attachment arm 500.

The attachment element 510 is arranged at a median plane XZ of the housing 204. In other words, the attachment element 510 is arranged at a vertical plane of symmetry of the housing 204.

As can be seen from the figures, the attachment element 510 can be arranged on the upper part of the housing 204. By "on the upper part of the housing" is meant "on the first quarter along the Z direction from the top of the housing 204". In particular, the attachment element 510 is arranged between 15% and 25% of the rear face 222 starting from the top 550 of the housing 204.

The attachment element 510 may be a lug. The lug protrudes from the rear face 222 of the housing 204.

Alternatively, the fastening element 510 is a magnet suitable for cooperating with another magnet placed on the fastening arm 500.

Alternatively, the fastening element 510 comprises an adhesive part suitable for cooperating with the fastening arm 500.

In an embodiment, the attachment arm 500 can be removable. In other words, the user can separate the attachment arm 500 from the attachment element 510 and thus from the case. In this way, the attachment arm 500 can be easily replaced, and another form of attachment arm can be used, for example.

Fastening arm 500 can be flexible. In other words, the fastening arm 500 can be deformed by the user or by the gravity of the device 100 placed in the toilet. As used herein, the term “flexible” refers to the ability of the fastening arm 500 to undergo elastic or plastic deformation under an applied load without breaking, such that the arm can bend, twist, or otherwise change shape to conform to the geometry of the toilet bowl or to accommodate installation forces. In certain embodiments, the flexibility is achieved through the selection of material (for example, a polymer, elastomer, or composite) and/or by incorporating structural features such as a reduced thickness, corrugations, or living hinges. This makes it easier to adapt the mounting arm to different toilet shapes, and ensures that the housing 204 touches the inner wall 112 of the toilet.

The attachment arm 500 can extend to a height along the Z axis of between 5 cm and 15 cm, in a resting configuration (i.e. neither stretched nor compressed). Fastening arm 500 may have a Y-axis width of between 1 cm and 2 cm. Attachment arm 500 is, for example, between 1 mm and 2 mm thick.

The collection port 218 and the fastening element 510 (or the fastening arm 500) can be arranged on either side of the urine analysis device 100, so that when the device 100 is held via the fastening element 510, the latter is at the top and the collection port 218 is at the bottom (relative to the vertical defined by gravity).

### STATION

A cleaning station 600 for a urine analysis device 100 is shown in FIG. 6. Cleaning station 600 is configured to receive urine analysis device 100. Together with the urine analysis device 100, the cleaning station 600 forms an assembly 700.

The cleaning station 600 comprises a container 610 and a charger 620. The container 610 is configured to receive a cleaning fluid 710. The container 610 is further dimensioned to at least partially receive the urine analysis device 100, so that the urine analysis device 100 is in contact with the cleaning fluid 710 when inserted into the container 610.

The charger 620 is configured to supply power to the urine analysis device 100 when the urine analysis device 100 is received at least partially in the container 610.

Assembly 700 is configured to switch from an operational configuration to a separate configuration, and vice versa.

In the operational configuration, the urine analysis device 100 is received in the container 610, as shown in FIGS. 7 (b), 8 (b), 10, 14 and 15.

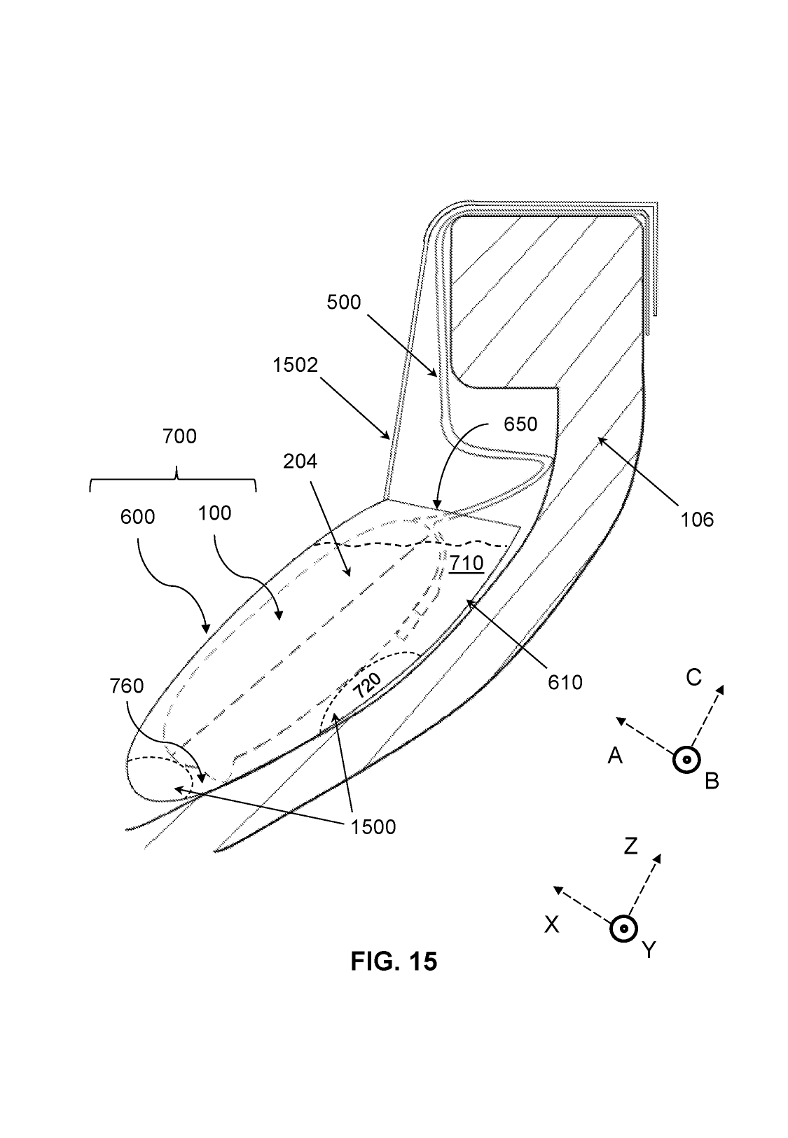

In an embodiment, shown in FIGS. 7 to 14, the urine analysis device 100 is away from the toilet bowl 106 in the operational configuration. Alternatively, shown in FIG. 15, the urine analysis device 100 is in position in the toilet bowl 106 in the operational configuration.

In the separated configuration, the urine analysis device 100 is away from the container 610. In particular, the device 100 is configured to be positioned entirely within the toilet bowl 106 in the separated configuration, as shown in FIG. 1.

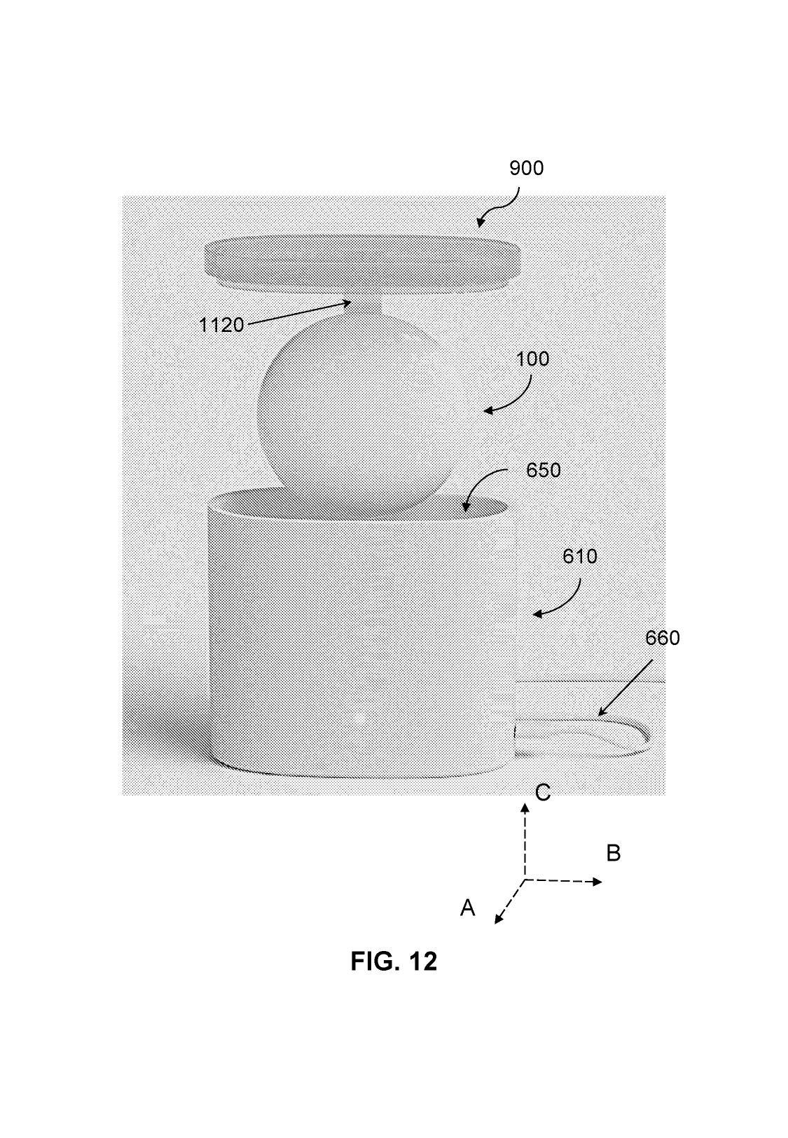

In FIG. 12, the device 100 is in transition between the operational configuration and the separated configuration.

Thus, the container 610 is configured to removably receive the urine analysis device 100, between the separate configuration and the operational configuration of the assembly 700. In the embodiments shown in FIGS. 7 to 14, the device 100 is inserted into the station 200 to pass into the operational configuration.

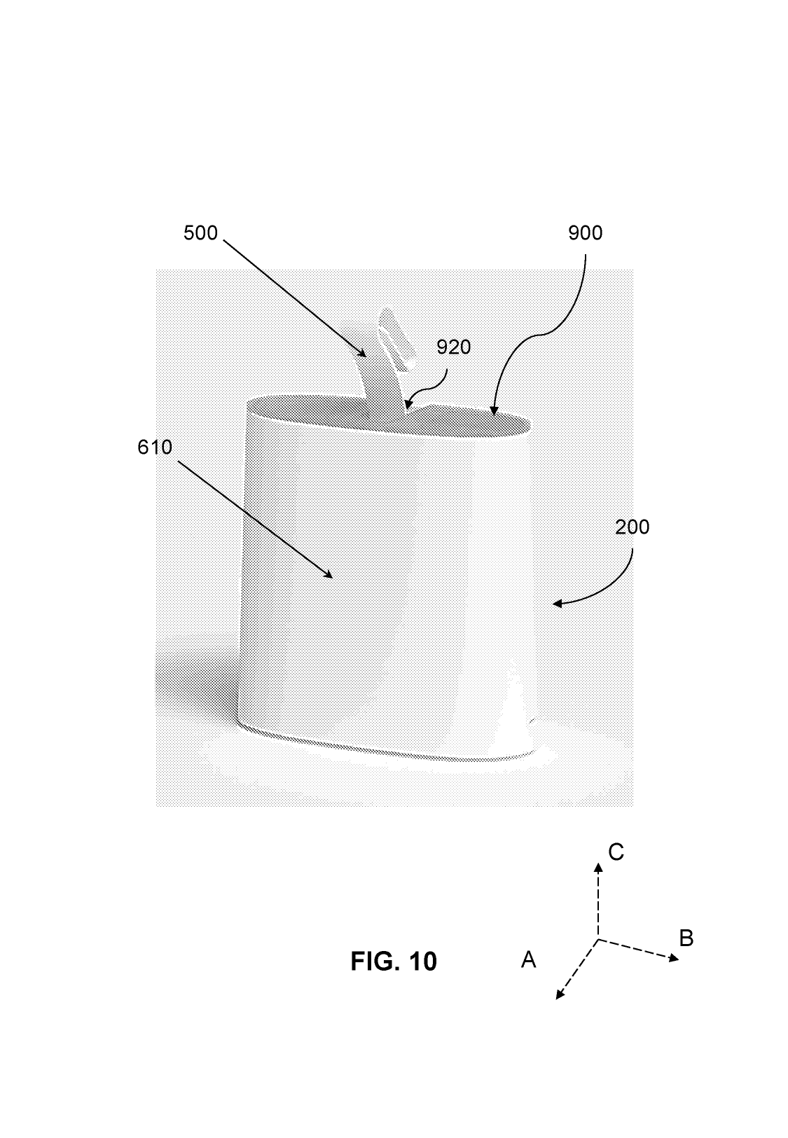

In the embodiment shown in FIG. 15, the station 200 is inserted around the device 100 in the bowl 106 to enter the operational configuration. The attachment arm 500 then protrudes from the container 610. The station 200 may then include a system for securing the container 610 to the bowl 106, such as one or more arms or a magnet. As used herein, a “system for securing” refers to any structural or functional element that maintains the container 610 in a desired position relative to the toilet bowl 106 and prevents unintended displacement during use. Such securing system may include, for example, one or more fasteners such as mechanical arms, clips, or brackets configured to grip or engage with the rim or wall of the toilet bowl; magnetic elements configured to cooperate with complementary magnets or ferromagnetic parts of the device or toilet bowl; suction cups or adhesive pads arranged on the container wall or bottom; frictional elements such as rubber feet; or a weight distribution system that stabilizes the container by lowering its center of gravity. In some embodiments, the securing system may be integral with the container wall or cover 900, while in other embodiments the securing means may be removable or adjustable to fit different toilet geometries. Alternatively, the container 610 rests directly on the housing 204 and on the fastening arm 500 of the device, in particular via the cover 900, in a manner similar to the embodiment shown in FIG. 10. In this case, station 200 may not include any means of attachment 1502 to bowl 106.

The purpose of cleaning station 600 is to clean the urine analysis device 100, mainly by cleaning fluid circuit 230. To this end, cleaning is carried out by introducing cleaning fluid 710 into fluid circuit 230 via collection port 218 by activating pump 780.

### Container shape

A directional reference frame ABC, with three axes A, B, C orthogonal in pairs, is used to describe the shape of the container.

Container 610 has a hollow shape. In particular, container 610 comprises a wall 630 which defines an internal volume 640. The internal volume 640 is accessible via an opening 650. Container 610 comprises a bottom 740, opposite opening 650, visible in FIG. 7.

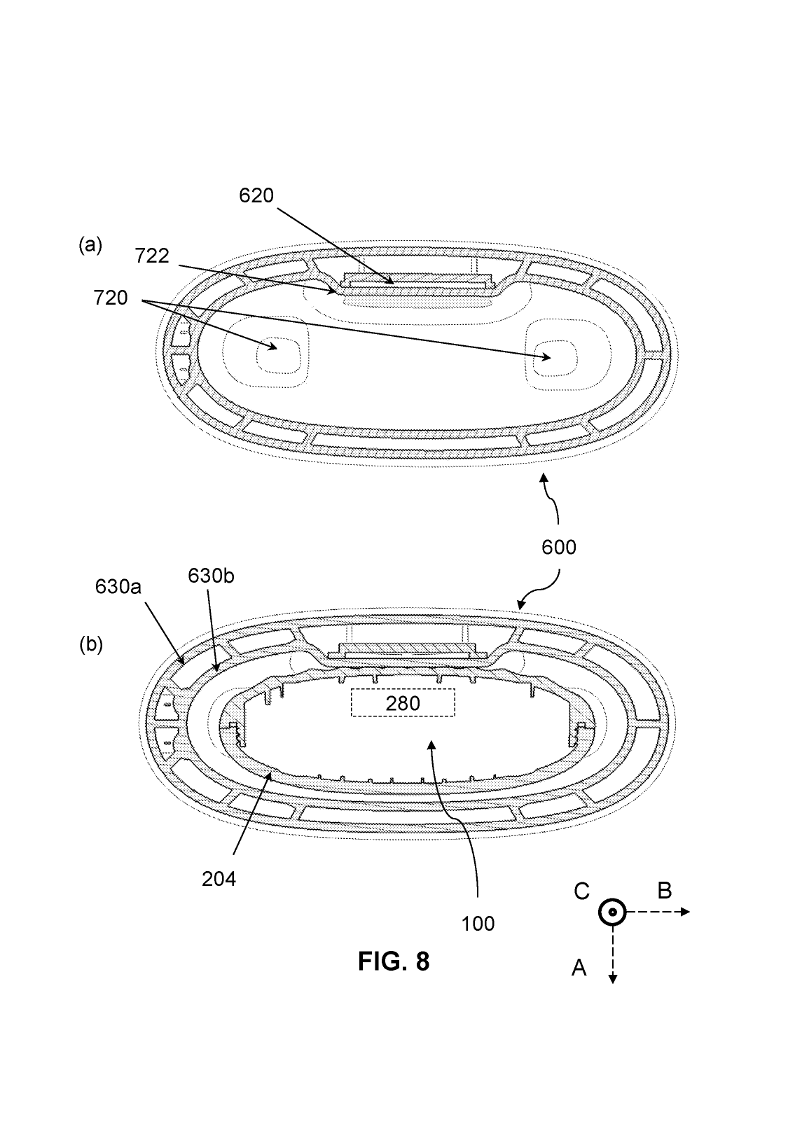

In an embodiment, visible in FIG. 8, wall 630 can be a double wall to stiffen container 610, with an outer wall 630a outside container 610 and an inner wall 630b inside container 610 defining inner volume 640. Alternatively, and not shown here, the wall 630 may consist of a single partition.

In an embodiment, as shown in FIGS. 6 to 10, container 610 has the shape of a truncated cone. In particular, the base of the cone (i.e. the part in contact with the support on which the cleaning station 600 is placed) has the largest cross-section. This shape improves the stability of the cleaning station 600, particularly when the internal volume 640 is filled with liquid. The base of the container is, for example, an oval or, more generally, a rounded convex shape (i.e. round, ovoid, ellipse, etc.). In a variant not shown, the base can be of any geometric shape such as a square, rectangle, triangle etc.

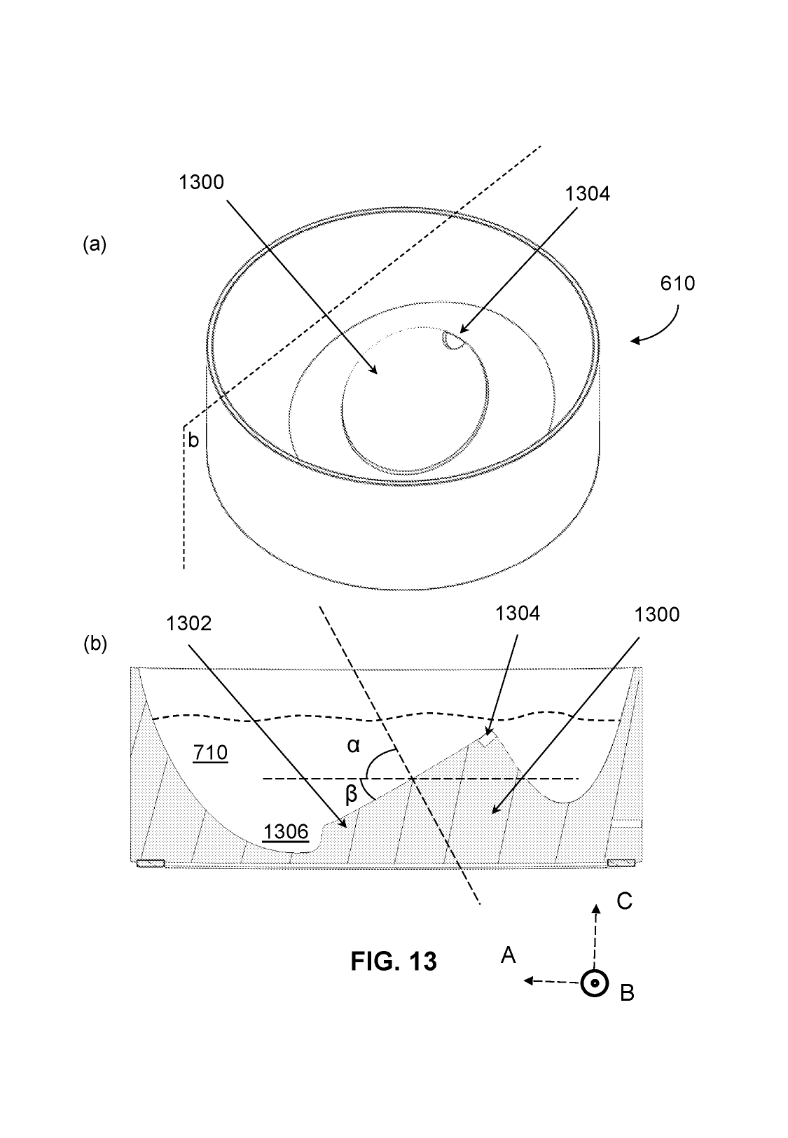

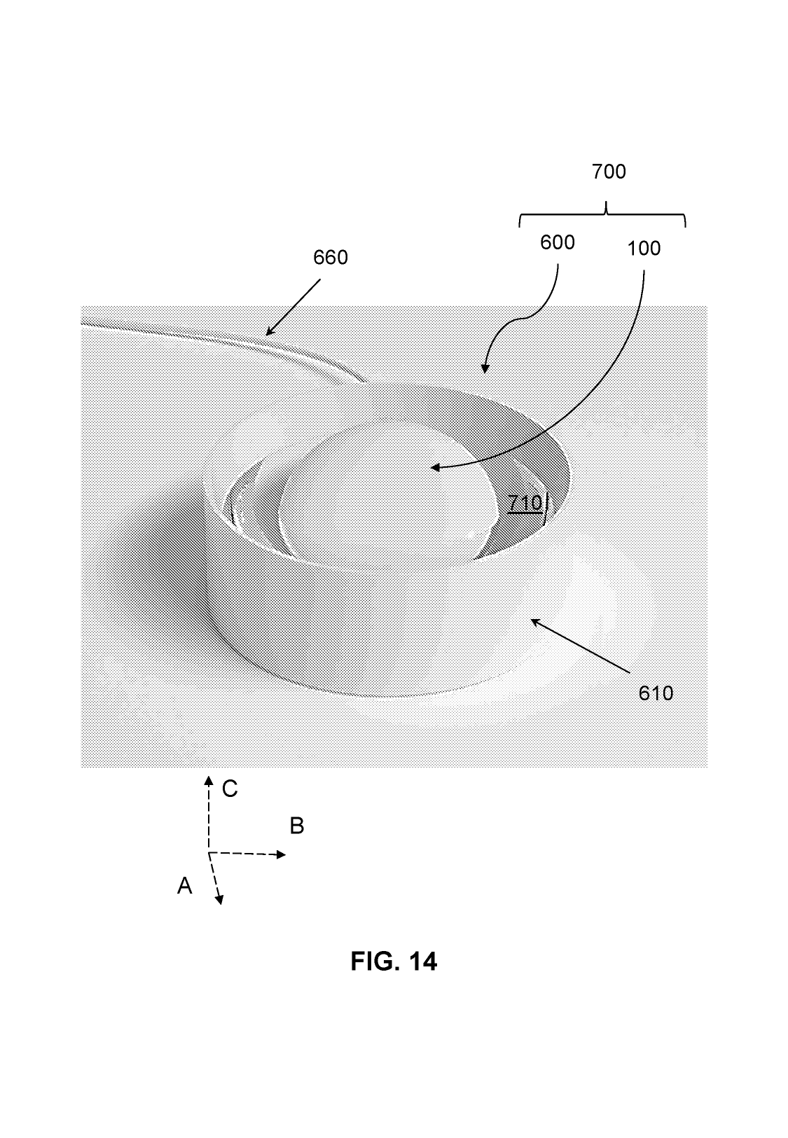

In another embodiment, as shown in FIGS. 12 to 14, the container 610 may have a straight cylindrical shape. For example, the base of the container is oval, as shown in FIG. 12. Alternatively, the container base may be round, as shown in FIGS. 13 and 14. Alternatively, the base can be of any geometric shape such as a square, a rectangle, a triangle etc.

In another embodiment, as shown in FIG. 15, the container 610 is complementary in shape to the inner surface of the bowl 106. In particular, container 610 may have an ovoid shape.

In an embodiment, as shown in FIGS. 6 to 12 and 15, the container 610 is taller than it is wide. In other words, the container extends over a height along the C axis that is greater than the transverse dimensions along the A or B axes. This elongated or vertical configuration facilitates insertion and removal of the device 100 by the user, and ensures that a sufficient depth of cleaning fluid can be maintained around the collection port 218 even with relatively small fluid volumes. The vertical form factor also allows the container to occupy a reduced footprint, making it more convenient to position on or near a toilet, countertop, or other limited-space environments. In particular, the height of the container along the C axis is, for example, between 10 cm and 30 cm. The length of the container along axis B is, for example, between 5 cm and 20 cm. The width of the container along axis A is, for example, between 3 cm and 10 cm. In some embodiments, the ratio of the height along axis C to the width along axis A may be between 2:1 and 6:1, or more particularly between 3:1 and 4:1, to achieve an optimal balance between stability, ergonomic handling, and cleaning performance. The container may also include structural reinforcements, such as ribs, a thickened base, or a weighted bottom portion, to further stabilize the taller geometry and prevent tipping.

Alternatively, as shown in FIGS. 13 and 14, container 610 is wider than it is high. In other words, the container extends over a height along the C axis that is less than the transverse dimensions along the A or B axes. This shape improves the stability of the cleaning station 600, particularly when the internal volume 640 is filled with liquid. In particular, the height of the container along the C axis is, for example, between 3 cm and 10 cm. The length of the container along the B axis is, for example, between 10 cm and 20 cm. The width of the container along A axis is, for example, between 10 cm and 20 cm.

In an embodiment, the urine analysis device 100 has a shorter dimension along the A axis. The container 610 is configured to receive the device 100 so that the A axis extends horizontally, parallel to the B axis of the container. The device 100 is thus received vertically along the C axis in the container 610.

In particular, the width of container 610 along axis A is substantially equal to the dimension of least length of device 100. Only a functional clearance is present between wall 630 and housing 204 of device 100 to allow insertion of device 100 into container 610. Thus, the device 100 is laterally supported by wall 630 to secure the device 100 in the container.

Alternatively, as shown in FIG. 14, container 610 is configured to receive device 100 so that axis A extends along a direction forming an angle α of between 20° and 70°, for example approximately 45° with horizontal direction X. In other words, the device 100 is received at an angle in the container 610, with the collection port 218 positioned at the bottom.

Container 610 is designed to form a clearance volume 760 around collection port 218. This clearance volume 760 is defined in particular by the fact that the collection port 218 is at a distance from the wall 630 and/or the bottom 740, in order to have a sufficient volume of cleaning fluid in the immediate vicinity of the collection port 218 for its suction.

The container 610 may comprise at least one protrusion 720, 722, 1300, 1500 configured to support the urine analysis device 100 in the operational configuration.

In particular, in an embodiment shown in FIGS. 7 and 8, the at least one protrusion 720 forms a support (or cradle) for holding the urine analysis device 100 in place in the cleaning station in the operational configuration. In particular, each cradle is designed to define the clearance volume 760 in the immediate vicinity of the collection port 218.

In particular, at least one protrusion 720 is arranged at the bottom of the container 610. In other words, the protrusion 720 is arranged on the bottom 740 or in the first quarter along the C axis starting from the bottom 740 of the container.

Beneficially, the device 100 is only supported by the protrusion(s) 720 in the container 610. In other words, the housing 204 is only in contact with the protrusions 720 and is not in contact with the rest of the wall 630.

In an embodiment visible in FIG. 7, the container 610 comprises at least two protrusions 720 arranged close to the bottom 740 of the container 610. According to one embodiment, the two protrusions 720 are arranged symmetrically on either side of the container along the B axis. As will be explained in greater detail later, the protrusions 720 leave the collection opening 218 free and unobstructed for the passage of the cleaning fluid 710. In particular, the protrusions 720 help define the clearance volume 760.

Alternatively or additionally, container 610 comprises at least one lateral protrusion 722 projecting substantially horizontally from wall 630. The lateral protrusion(s) 720 wedge the device 100 laterally in the container 610 and hold it in position.

In an embodiment shown in FIG. 13, the container 610 comprises at least one protrusion 1300 in the form of a promontory configured to support the device 100. In particular, the protrusion 1300 comprises a receiving surface 1302 configured to contact one of the two faces 220, 222 of the housing 204 in order to support the device 100. The receiving surface 1302 extends in a plane forming an angle β with the horizontal direction of between 0° and 70°. Beneficially, the receiving surface 1302 is complementary in shape to the face 220, 222 of the housing 204 with which it is in contact. In particular, the protrusion 720 may comprise at least one retaining element 1304 configured to cooperate with the face 220, 222 of the housing so as to hold it in position. As shown in FIG. 13, the retaining element 1304 is, for example, a notch of complementary shape with at least one part of the device 100, in particular with the attachment element 510 of the housing 204.

Similar to the container 610 of FIG. 7, the container 610 of FIG. 13 defines a clearance volume 1306 within which the collection port 218 is located when the device 100 is in its operational position.

In the embodiments of FIGS. 7 and 13, due to the positioning of the collection port 218 on the housing 204 and the positioning of the device 100 in the cleaning station 600, the clearance volume 1306 is at the level of the bottom of the container 610.

### Cover

The cleaning station 600 can include a cover 900 configured to at least partially close the internal volume 640 of the container 610. In other words, the cover 900 is configured to at least partially obstruct the opening 650 of the internal volume 640.

Cover 900 is configured to move from an open configuration to a closed configuration, and vice versa. In the open configuration, cover 900 is away from opening 650. The open configuration makes it easy to insert or remove the device 100 from the container 610. In the closed configuration, cover 900 cooperates mechanically with container 610 to at least partially close opening 650. The closed configuration keeps the device 100 inside the container 610.

Cover 900 is made of a plastic material, for example. As used herein, a “plastic material” refers to a polymeric composition that can be molded, extruded, or otherwise shaped, and that provides mechanical strength, dimensional stability, and resistance to moisture in the intended toilet-bowl environment. Suitable plastic materials may include thermoplastics such as polycarbonate (PC), polyethylene terephthalate (PET), acrylonitrile butadiene styrene (ABS), polypropylene (PP), or polyethylene (PE), as well as thermosetting resins or reinforced composites. In some embodiments, the plastic material may further comprise additives such as pigments, fillers, UV stabilizers, antimicrobial agents, or hydrophobic/hydrophilic surface treatments to enhance durability, user safety, and ease of cleaning. The cover may be at least partially transparent or translucent, so that the user can see at least partially through the device 100 through the cover 900 when the assembly is in the operational configuration and the cover 900 in the closed configuration.

The cover 900 may have a shape substantially complementary to the opening 650. In an embodiment, the cover 900 may snap into place at the opening 650. Alternatively, cover 900 can be magnetized to container 610. Alternatively, cover 900 can be hinged to container 610.

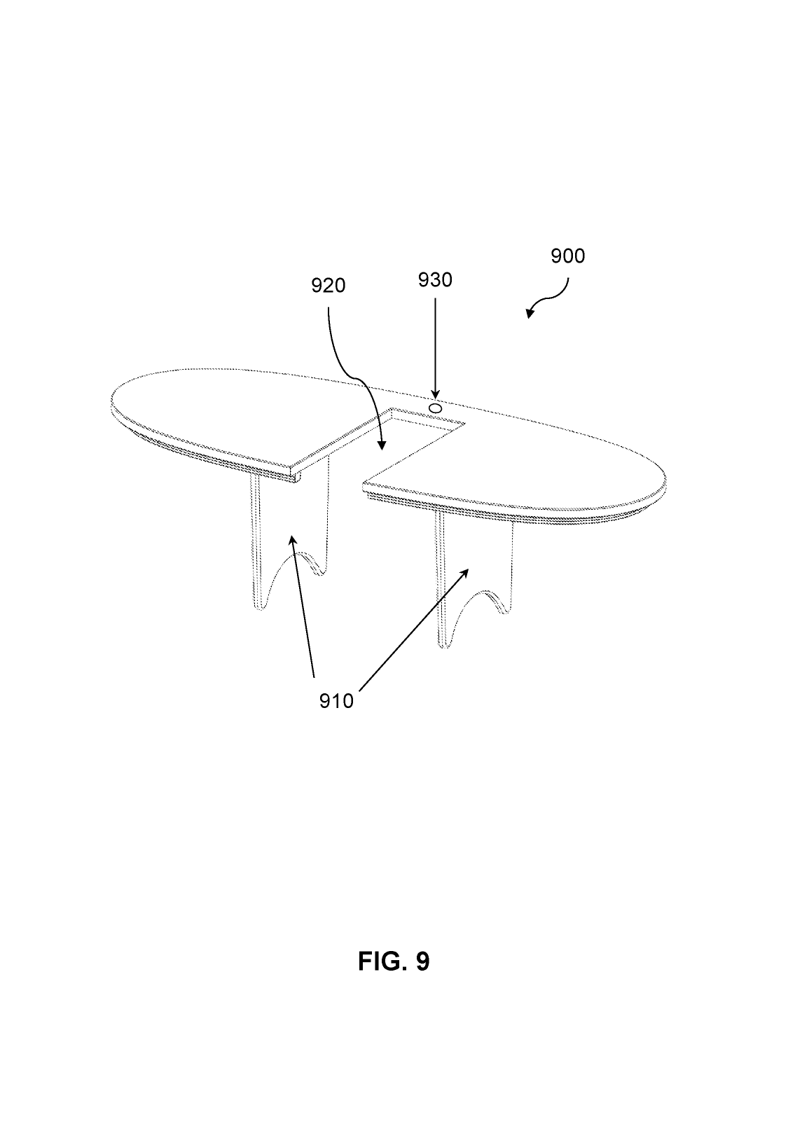

With reference to FIG. 9, the cover 900 may comprise at least one wedge 910 projecting from the cover 900 towards the internal volume 640 of the container 610 in the closed configuration. Each wedge 910 is configured to hold the device 100 in position in the container 610. In particular, each wedge 910 is sized to contact the housing 204 in the closed configuration of the cover 900 (a slight functional clearance may be provided). Each wedge 910 may comprise an end of complementary shape with housing 204. In the operational configuration of the assembly and in the closed configuration of the cover, the device 100 is then held in position by the at least one protrusion 720 and the at least one wedge 910. As shown in FIG. 9, the cover may comprise two wedges 910.

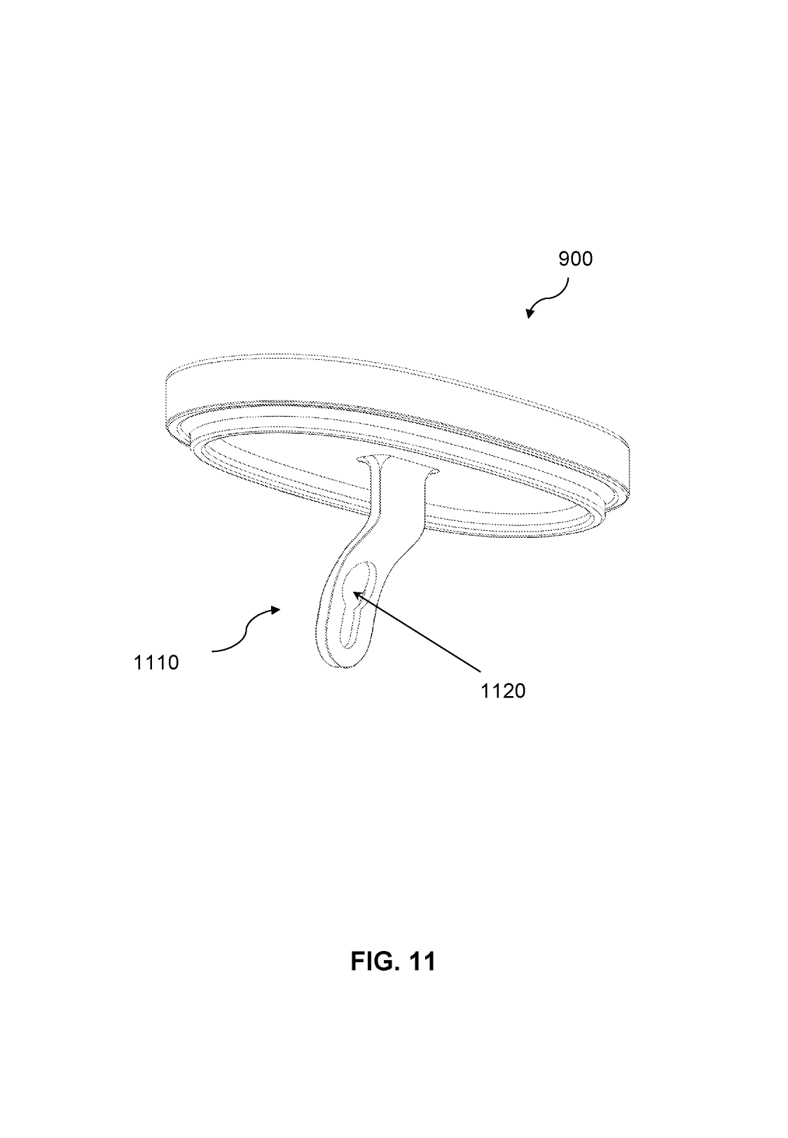

Alternatively, as shown in FIG. 11, the cover 900 may comprise a hook 1110 configured to cooperate with the attachment element 510 of the urine analysis device 100. The hook 1110 projects towards the internal volume 640 in the closed configuration of the cover 900. When the attachment element 510 is a pin, the hook may comprise an opening 1120 suitable for receiving the pin. Alternatively, when the attachment element 510 is a magnet, the hook 1110 may comprise a complementary magnet to link to the magnet of the device 100. As shown in FIG. 12, the device 100 can thus be linked to the cover 900 in the open configuration of the cover and in the separate configuration of the assembly. The device 100 can then be easily inserted into the container 610 at the same time as the cover 900 is placed over the opening 650, so that the cover 900 moves into the closed configuration at the same time as the assembly moves into the operational configuration.

In an embodiment, as shown in FIG. 9, cover 900 may comprise at least one through opening 920. The through opening 920 is suitable for being passed through by a fastening arm 500 of the urine analysis device 100, when the assembly 700 is in the operational configuration and when the cover 900 is in the closed configuration, as seen in FIG. 10. Fastening arm 500 thus protrudes at least in part from container 610 in the operational configuration. Container 610 may include a stud 670 projecting from the rim of opening 650. The through opening 920 is complementary in shape to the stud 670. In particular, the stud 670 allows the user to orientate himself in the direction in which to close the cover 900 over the opening 650.

The through opening 920 eliminates the need to remove the fastening arm 500 when the device 100 is placed in the container 610. The user can therefore easily manipulate the device 100 by grasping the arm to insert or remove it from the container 610. The attachment arm 500, which does not generally receive urine in the bowl, can be better suited for gripping by a user. In this way, the user can move the device 100 from the toilet to the cleaning station without touching the urine-receiving housing 204. In addition, the proximal part of the attachment arm 500 (the part close to the housing 204) can be washed in the cleaning station 600.

Alternatively, as shown in FIG. 11, cover 900 does not have an opening. In other words, cover 900 is dimensioned to completely obstruct opening 650 of container 610 in the closed configuration.

Alternatively, or additionally, the cover 900 may comprise a thinner zone 930. The thinner zone 930 makes the indicator light 290 of the device 100, in particular the battery 280 charge indicator of the device 100, visible to the user through the cover 900. For example, the thinner zone 930 has a thickness of less than 2 mm. The thinner zone 930 has, for example, the shape of a circle with a diameter of less than 1 cm.

### CLEANING FUNCTION - station side

As explained above, the container 610 is dimensioned to at least partially receive the urine analysis device 100, so that the urine analysis device 100 is in contact with the cleaning fluid 710 when it is inserted into the container 610.

In particular, as visible in FIG. 7 (b), the container 610 and the at least one protrusion 720, 722, 1300, 1500 are designed so that the collection port 218 is immersed in the cleaning fluid 710 in the operational configuration.

In particular, the container 610 is further dimensioned, in particular with the at least one protrusion 720, 722, 1300, to define the clearance volume 760. This volume is defined in particular by the fact that the collection port 218 is at a distance from the wall 630 and/or the bottom 740 of the container 610. By "away", it is meant at least 3 mm, in particular at least 5 mm, between the collection port 218 and the furthest of the wall 630 or the bottom 740. In certain preferred embodiments, the distance may range between 5 mm and 20 mm, and for example not more than 30 mm, which has been found to provide sufficient volume of cleaning fluid in the immediate vicinity of the collection port while avoiding unnecessary bulk or excessive fluid requirements. As will be explained below, this thus enables unimpeded pumping of the cleaning fluid 710 by the device 100 to enable its cleaning from the inside, in particular the cleaning of the fluidic circuit 230.

In an embodiment, the container 610 is dimensioned so that the housing 204 is completely submerged under the cleaning fluid 710 in the operational configuration. In this way, the entire exterior of housing 204 can be passively cleaned by contact with cleaning fluid 710. The housing 204 is regularly in contact with urine, which can make it dirty and cause hygiene problems. The cleaning station 600 therefore enables at least partial cleaning of the outside of the housing 204 of the device 100 passively.

In an embodiment, cleaning station 600 has no system for circulating cleaning fluid 710 in container 610. The cleaning station 600 is thus a passive system for cleaning, the device 100 being the element actively circulating the cleaning fluid 710. The movement of the fluid by the device 100 also facilitates cleaning of the exterior of the housing 204.

Alternatively or additionally, the cleaning station 600 may include a pump configured to circulate the cleaning fluid 710 in the container 610 and inject the fluid into the device 100. Alternatively, the pump may simply generate a circulation of cleaning fluid in the container 610 to promote cleaning of the housing 204.

To ensure that the user has put in a sufficient quantity of cleaning fluid, the container 610 may include a gauge 750 indicating the recommended volume of cleaning fluid 710 to be poured into the container 610 when the device 100 is not inserted into the container 610. The gauge 750 is, for example, a horizontal mark placed on the wall 630, in particular inside the internal volume 640.

The cleaning fluid 710 is in particular a detergent, for example Tergazyme ®. Alternatively, the cleaning fluid is water. The cleaning fluid can be generated by mixing a (solid) detergent tablet with water, directly in container 610.

### CLEANING FUNCTION - device side

Device 100 is configured to switch from a urine analysis configuration to a cleaning configuration, and vice versa. In the urine analysis configuration, the device 100 is configured to be placed in the toilet bowl 106, to collect and analyze a user's urine, as shown in FIG. 1 and explained in detail above.

The device 100 is configured to switch to the cleaning configuration when the device 100 is placed in the container 610, in the operational configuration of the assembly 700. The urine analysis device 100 is then configured to initiate a cleaning phase in which fluid circuit 230 is in the sampling position. Device 100 is then configured to aspirate cleaning fluid 710 present in container 610, via collection port 218 by means of pump 780 of fluidic circuit 230. Device 100 is configured to circulate cleaning fluid 710 in fluid circuit 230 and to drain cleaning fluid 710 through drain port 530. A cleaning phase may comprise a plurality of successive suction and purge cycles.

In particular, in the cleaning configuration, fluidic circuit 230 comprises in series the collection port 218, the injector and the drain port 530. Fluidic circuit 230 thus communicates between collection port 218 and drain port 530. During the cleaning phase, cleaning fluid 710 passes through fluidic circuit 230, from the collection port 218 to the drain port 530, via the injector, thus enabling them to be cleaned.

During operation of device 100 in the urine analysis configuration, urine flows repeatedly through fluid circuit 230. Deposits can then build up, leading to partial obstruction of the fluidic circuit and/or contamination of subsequent urine, and thus distorting future analyses. Cleaning of the fluidic circuit 230 of the device 100 by the cleaning station 600 enables these deposits to be removed, thereby increasing the service lifespan of the device 100 and making analyses more reliable.

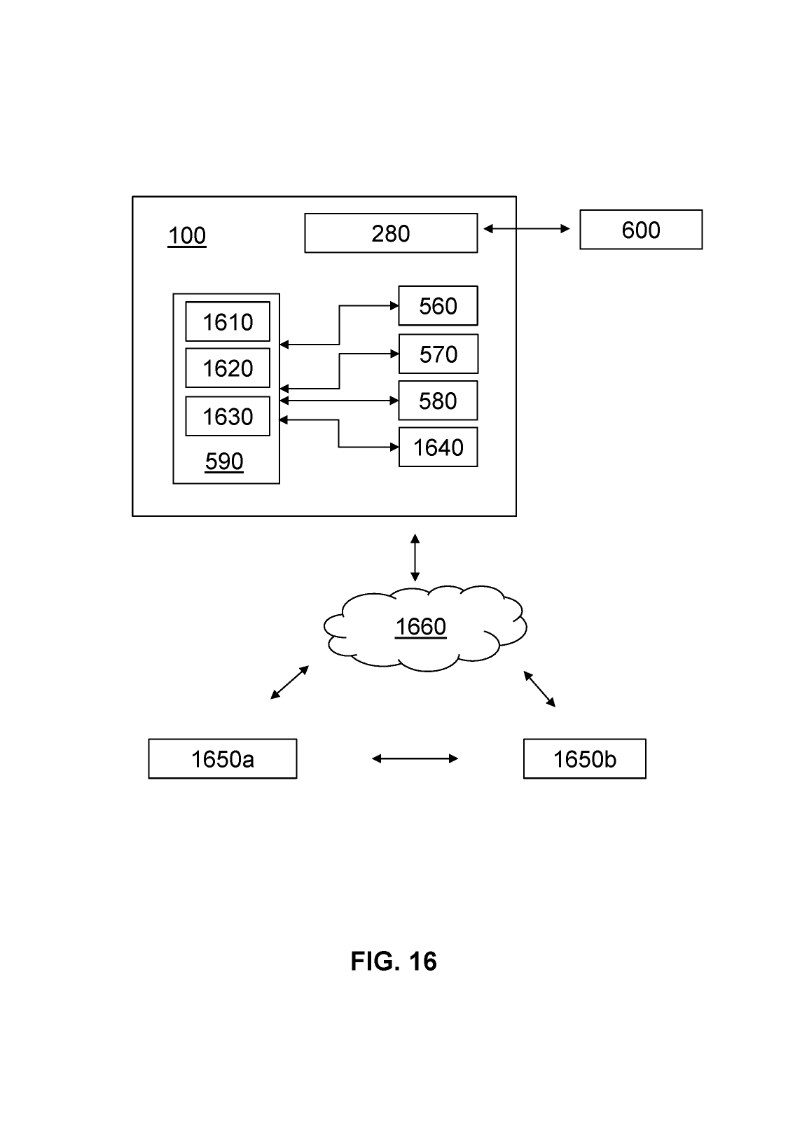

In an embodiment, the cleaning phase is controlled by a control circuitry 590 of the device 100, shown in greater detail in FIG. 16. Control circuitry 590 comprises a processor 1610, a memory 1620 and an I/O interface 1630 (input/output) configured to send and receive data from control circuitry 590. A communication module 1640 can be provided to exchange data with an external terminal 1650. The communication module 310 can be a wireless module, such as Wi-Fi, Bluetooth®, Bluetooth Low Emission®, etc. The control circuitry 590 can in particular exchange with the radar sensor 570, the temperature sensor 560 and/or the liquid sensor 580 to control the cleaning phase. The term “control circuitry” as used herein refers to one or more microcontrollers, processors, or equivalent computing devices, together with associated memory and interfaces, adapted to execute stored instructions and manage the operation of the station.

The power supply system 280 is configured to supply these components with energy.

Memory 1620 can store instructions which, when executed by processor 1610, implement the method(s) of the present description. The methods are for example performed locally, by the processor 1610 of the device 100. When executed by the processor 1610, these programs control actuation of pumps, motors, and sensors, acquisition from the analyzer, as well as one or more other devices described herein.

The device 100 can communicate, using the communication module 1640 and using a communication network 1660, with an external terminal 1650, such as a mobile terminal 1650a ("smartphone"). The device 100 can also communicate with a server 1650b, either directly via the communication network 1660 or via the external mobile terminal 1650a.

In particular, the control circuitry 590 is configured to activate the pump 780 when the device 100 is placed in the container 610 in order to aspirate the cleaning fluid 710 in cleaning configuration. The control circuitry 590 is configured to control the power supply to the pump 780 by the power supply device 280 of the device 100.

The cleaning configuration of device 100 can be activated by means of an activation signal sent by the user via an external terminal 1650, for example via a smartphone or a server.

In an embodiment, the control circuitry 590 is configured to activate the pump 780 when the control circuitry 590 determines that predetermined cleaning conditions are met. The cleaning conditions include, for example, receiving a signal from the power supply system 280 indicating that it is receiving power from the charger 620. Alternatively or additionally, the cleaning conditions include receipt of a signal sent by the radar sensor 570 indicating that the wall 630 of the container 610 is detected. Alternatively or additionally, the cleaning conditions include receiving a signal from temperature sensor 560 indicating that a temperature change on housing 204 has been detected, indicating the presence of cleaning fluid 710 in container 610. Alternatively or additionally, the cleaning conditions include the reception of a signal from the external terminal 1650 (e.g. a user triggering a cleaning session).

In an embodiment, the control circuitry 590 is configured to suspend operation of the pump 780 when the control circuitry 590 receives a signal from the liquid sensor indicating that the fluid drawn in is gas or mixed fluid, and not liquid, indicating an absence or lack of cleaning fluid 710 in the container 610.

### CHARGING FUNCTION - station side

Charger 620 is configured to supply power to urine analysis device 100 when assembly 700 is in operational configuration. Charging of the device 100 can take place as soon as the device 100 is inserted into the container 610. Alternatively, station 600 may include a user-operated control button to start or stop charging of device 100.

The charger 620 is housed in the container 610. In other words, the charger 620 is housed inside the internal volume 640 of the container, or the charger 620 is housed between the two partitions forming the wall 630 of the container 610.

The charger 620 is positioned opposite the wall 630 of the container 610. As shown in FIG. 8, charger 620 can be positioned in the lateral protrusion 722 formed in wall 630. The charger 620 is positioned, along the depth of the container 610 along the C axis, at least partly between the bottom 740 of the container 610 and the gauge 750.

In an embodiment, the charger 620 is a wireless charger, i.e. charging between the charger 620 and the device 100 is wireless. In other words, there is no physical electrical connection connecting the device 100 to the charger 620. In particular, the charger 620 is an electromagnetic induction charger. In particular, the charger 620 comprises a coil. The inventors noted that wireless charging was possible despite the presence of cleaning fluid in the container and therefore between wall 630 and device 100.

In a variant not shown, the charger 620 comprises a charging port connectable to the urine analysis device 100, for example a USB port protruding from wall 630 into the internal volume 640.

The cleaning station 600 may include an electrical connection cable 660 for connecting the charger 620 to an electrical network. The cable 660 protrudes, for example, from the bottom of the outside of the container 620. Cable 660 may be removable, for example via a USB connection.

Alternatively or additionally, the cleaning station 600 comprises a battery configured to power the charger 620. The battery can be recharged via the electrical connection cable 660. Alternatively, the battery is in the form of replaceable cells.

### CHARGING FUNCTION - device side

In an embodiment, the power supply system 280 is powered by the charger 620, whether or not there is cleaning fluid in the container 610. The cleaning station 600 thus functions as a simple charger.

In an embodiment, the power supply system 280 is configured to be supplied with power by the charger 620 and to simultaneously supply power to the device 100. In this way, activation of the pump 780 can be powered by energy ultimately supplied by the charger 620 and be done without discharging the battery (and thus without reducing the duration of use of the device 100 in the toilet). What's more, when the pump's consumption is lower than the charger's power, the power supply system 280 can charge the battery, despite the fact that the pump 780 is cleaning the fluid circuit.

The battery is arranged in the device 100 so as to face the charger 620 in the operational configuration. In particular, the battery is positioned opposite the face of the housing 204 in contact with the lateral protrusion 720 housing the charger 620.

When assembly 700 is in operational configuration, indicator light 290 of device 100 is configured to provide visual information about the battery's state of charge. This information can be observed by the user, enabling him to know when the battery charge is complete.

In an embodiment, the power supply system 280 comprises no energy storage means. The energy supplied by the charger 620 is then directly transmitted to the operation of the device 100, in particular to the pump.

### METHOD

A method for cleaning device 100 using cleaning station 600 will now be described.

Assembly 700 is initially in the separate configuration. The urine analysis device 100 is placed, for example, in the user's toilet bowl 106, as shown in FIG. 1. The device 100 is then in the urine analysis configuration and can perform regular analyses of the user's urine.

Then, when for example the cartridge 202 is used up or the housing 204 appears soiled or the battery charge level is low, the user may decide to clean the device 100.

The user fills container 620 with cleaning fluid 710, typically up to gauge 750.

In an embodiment, the user then removes the device 100 from the toilet, for example by gripping the fastening arm 500, and inserts the device 100 into the container 620, as shown in FIG. 10. Alternatively, the user inserts the container 610 around the device 100 in the toilet bowl 106, as shown in FIG. 15.

The device 100 is then at least partially immersed in the cleaning fluid 710. In particular, the collection port 218 is immersed in the cleaning fluid 710. The user can also close the container 610 with the cover 900. The assembly 700 then switches from the open configuration to the operational configuration. Cover 900 changes from open to closed configuration.

The part of the housing 204 in contact with the cleaning fluid 710, and if applicable, the fastening arm 500, are then passively cleaned.

The charger 620 supplies the device 100 with power, in particular the power supply system 280. In an embodiment, the charger 620 supplies the device 100 wirelessly, by electromagnetic induction. The power supply system 280 may comprise a battery, which is then recharged. The user can monitor the battery's charge status via the device 100's indicator light 290, visible through the cover 900.

In parallel or successively, the user sends an activation signal via an external terminal 1650 to the device 100 to switch it from the urine analysis configuration to the cleaning configuration.

The control circuitry 590 of device 100 then initiates a first cycle of the cleaning phase by activating the pump with energy supplied by charger 620 and/or the battery of device 100 when predetermined cleaning conditions are met. In particular, the cleaning conditions include receipt of a signal sent by the power supply system 280 indicating that it is receiving energy from the charger 620 and/or receipt of a signal sent by the radar sensor 570 indicating that the wall 630 of the container 610 is detected, and/or receipt of a signal sent by the temperature sensor 560 indicating that a temperature change on the housing 204 has been detected, indicating the presence of cleaning fluid 710 in the container 610.

The pump then draws in cleaning fluid 710 through collection port 218. Cleaning fluid 710 circulates and cleans fluid circuit 230 until it is drained through drain port 530.

The control circuitry 590 suspends pump operation if the control circuitry 590 receives a signal from the liquid sensor indicating that the fluid drawn in is gas or mixed fluid, and not liquid, indicating an absence or lack of cleaning fluid 710 in the container 610.

The cleaning phase may comprise several cycles of pumping and draining the cleaning fluid 710. The cleaning phase may stop after a predetermined time, for example between five minutes and five hours, or when the user removes the device 100 from the container 610, for example when the battery charge is complete.

Cleaning and charging the urine analysis device 100 is therefore simple and intuitive. All the user has to do is insert the device 100 into the container 610, so that the charger 620 can supply power to the device 100 and the cleaning fluid 710 can clean the outside of at least part of the housing 204. The device 100 can also draw in cleaning fluid 710 and circulate it in fluid circuit 230 to clean it. The cleaning station 600 can therefore be used to clean the outside and inside of the device 100, as well as to recharge it, thereby improving the service life and quality of the measurements made by the device 100.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles "a" and "an" may be employed in connection with various elements and components, processes or structures described herein. This is merely for convenience and to give a general sense of the compositions, processes or structures. Such a description includes "one or at least one" of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A cleaning station for a urine analysis device, the cleaning station comprising:
a container configured to receive a cleaning fluid, the container being further dimensioned to at least partially receive the urine analysis device, so that the urine analysis device is in contact with the cleaning fluid,
a charger configured to supply power to the urine analysis device when the urine analysis device is at least partially received in the container.

2. The cleaning station according to claim 1, wherein the urine analysis device comprises a rechargeable battery and the charger is configured to electrically charge the battery.

3. The cleaning station according to claim 1, wherein the charger is a wireless charger, so that charging between the charger and the urine analysis device is wireless.

4. The cleaning station according to claim 1, wherein the container comprises at least one protrusion configured to support the urine analysis device.

5. The cleaning station according to claim 4, wherein the device is solely supported by the at least one protrusion in the container.

6. The cleaning station according to claim 4, wherein the at least one protrusion defines a clearance volume intended to receive the collection port of the analysis device.

7. The cleaning station according to claim 1, wherein the cleaning station comprises a cover configured to at least partially close the container.

8. The cleaning station according to claim 7, wherein the cover comprises at least one wedge projecting from the cover towards the container, each wedge being configured to hold the urine analysis device in position in the container.

9. The cleaning station according to claim 7, wherein the cover comprises at least one aperture adapted to be passed through by an attachment arm of the urine analysis device.

10. An assembly comprising:
a urine analysis device, the urine analysis device comprising a housing and a fluidic circuit housed within the housing and configured to circulate urine in the urine analysis device, the fluidic circuit comprising a collection port on the housing,
a station according to claim 1,
the assembly being configured to switch
from an operational configuration in which the urine analysis device is received in the container to
a separate configuration in which the urine analysis device is away from the container.

11. The assembly of claim 10, wherein the container is sized so that the collection port is immersed in the cleaning fluid in the operational configuration.

12. The assembly according to claim 10, wherein the container is formed by a wall and a bottom, the container being dimensioned so that the collection port is spaced apart from at least one of the wall and the bottom.

13. The assembly according to claim 10, wherein the fluidic circuit comprises a pump configured to draw liquid into the fluidic circuit via the collection port.

14. The assembly as claimed in claim 13, wherein the charger is configured to, in an operational configuration, power the pump of the fluidic circuit, such that cleaning fluid is drawn into the fluidic circuit.

15. The assembly according to claim 10, wherein the charger is further configured to recharge a battery of the urine analysis device.

16. The assembly according to claim 10, wherein the urine analysis device is configured to switch from a urine analysis configuration to a cleaning configuration, the device comprising at least one reagent suitable for reacting with urine, the fluidic circuit comprising a drainage port on the housing,
the fluidic circuit being configured, in the urine analysis configuration, to inject the fluid collected by the collection port onto the reagent,
the fluidic circuit being configured, in the cleaning configuration, to circulate the fluid collected by the collection port directly to the drain port.

17. A cleaning and charging method implemented by an assembly according to claim 10, the method comprising the following steps, after setting the assembly from the separated configuration to the operational configuration:
powering the device by the charger,
cleaning the device with the cleaning fluid in the container.

18. A cleaning station for a urine analysis device having a housing, a collection port, and a pump, the cleaning station comprising:
a container having a wall and a bottom that define an internal volume accessible through an opening and configured to receive a cleaning fluid;
at least one support feature arranged within the internal volume to position a received urine analysis device such that the collection port is immersed in the cleaning fluid and lies within a clearance pocket spaced from the wall and/or the bottom by at least 3 mm to permit unimpeded intake of the cleaning fluid through the collection port; and
a wireless charger comprising an inductive coil disposed in or adjacent the wall, the charger being positioned along a depth of the container at least partly between the bottom and a fill gauge and being configured to transfer power to the received urine analysis device while the collection port is immersed;
wherein the cleaning station lacks any pump configured to circulate the cleaning fluid within the container, so that circulation through the device occurs by operation of the pump of the received urine analysis device when powered by the charger.

19. The cleaning station according to claim 18, wherein the clearance pocket spaces the collection port from the wall and/or the bottom by at least 5 mm and not more than 30 mm.

20. The cleaning station of claim 18, wherein the at least one support feature comprises a lateral protrusion projecting from the wall to laterally wedge the housing.
